(19) United States
(12) Patent Application Publication
(10) Pub. No.: US 2003/0195187 A1
(43) Pub. Date: Oct. 16, 2003

(54) Guanidino compounds

(75) Inventors: Rustum S. Boyce, San Francisco, CA (US); Daniel Chu, Santa Clara, CA (US)

Correspondence Address: FOLEY AND LARDNER, SUITE 500, 3000 K STREET NW, WASHINGTON, DC, 20007, US

(73) Assignee: CHIRON CORPORATION

(21) Appl. No.: 10/351,574

(22) Filed: Jan. 27, 2003

Related U.S. Application Data
(60) Provisional application No. 60/353,188, filed on Feb. 4, 2002.

(51) Int. Cl. A61K 31/55; A61K 31/47; A61K 31/198; C07D 217/12; C07D 223/16

(52) U.S. Cl. 514/217.01; 514/310; 514/563; 540/593; 546/146

(57) ABSTRACT

A variety of small, guanidino group-containing molecules capable of acting as MC4-R agonists are provided. The compounds have various structures provided herein. The compounds are useful in treating MC4-R mediated diseases and may be formulated into pharmaceutical formulations and compositions.

### CROSS REFERENCES TO RELATED APPLICATIONS

This application claims priority to U.S. Provisional Application No. 60/353,188 filed on Feb. 4, 2002, the entire disclosure of which is hereby incorporated by reference in its entirety and for all purposes.

### FIELD OF THE INVENTION

This invention relates to melanocortin-4 receptor (MC4-R) agonists and methods of their preparation. The invention also relates to methods of treating melanocortin-4 receptor-mediated diseases, such as obesity or diabetes, by activating the melanocortin-4 receptor with compounds provided herein.

### BACKGROUND OF THE INVENTION

Melanocortins are peptide products resulting from post-translational processing of pro-opiomelanocortin and are known to have a broad array of physiological activities. The natural melanocortins include the different types of melanocyte stimulating hormone (α-MSH, β-MSH, γ-MSH) and ACTH. Of these, α-MSH and ACTH are considered to be the main endogenous melanocortins.

The melanocortins mediate their effects through melanocortin receptors (MC-Rs), a subfamily of G-protein coupled receptors. There are at least five different receptor subtypes (MC1-R to MC5-R). MC1-R mediates pigmentation of the hair and skin. MC2-R mediates the effects of ACTH on steroidogenesis in the adrenal gland. MC3-R and MC4-R are predominantly expressed in the brain. MC5-R is considered to have a role in the exocrine gland system.

The melanocortin-4 receptor (MC4-R) is a seven-transmembrane receptor. MC4-R may participate in modulating the flow of visual and sensory information, coordinate aspects of somatomotor control, and/or participate in the modulation of autonomic outflow to the heart. K. G. Mountjoy et al., Science, 257:1248-125 (1992). Significantly, inactivation of this receptor by gene targeting has resulted in mice that develop a maturity onset obesity syndrome associated with hyperphagia, hyperinsulinemia, and hyperglycemia. D. Husznar et al., Cell, 88(1): 131-41 (1997). MC4-R has also been implicated in other disease states including erectile disorders, cardiovascular disorders, neuronal injuries or disorders, inflammation, fever, cognitive disorders, and sexual behavior disorders. M. E. Hadley and C. Haskell-Luevano, The proopiomelanocortin system, Ann. N. Y. Acad. Sci., 885:1 (1999).

Furthermore, observations in connection with endogenous MCx-R antagonists indicate that MC4-R is implicated in endogenous energy regulation. For example, an agouti protein is normally expressed in the skin and is an antagonist of the cutaneous MC receptor involved in pigmentation, MC1-R. M. M. Ollmann et al., Science, 278:135-138 (1997). However, overexpression of agouti protein in mice leads to a yellow coat color due to antagonism of MC1-R and increased food intake and body weight due to antagonism of MC4-R. L. L. Kiefer et al., Biochemistry, 36: 2084-2090 (1997); D. S. Lu et al., Nature, 371:799-802 (1994). Agouti related protein (AGRP), an agouti protein homologue, antagonizes MC4-R but not MC1-R. T. M. Fong et al., Biochem. Biophys. Res. Commun. 237:629-631 (1997). Administration of AGRP in mice increases food intake and causes obesity but does not alter pigmentation. M. Rossi et al., Endocrinology, 139:4428-4431 (1998). Together, this research indicates that MC4-R participates in energy regulation, and therefore, identifies this receptor as a target for a rational drug design for the treatment of obesity.

In connection with MC4-R and its uncovered role in the etiology of obesity and food intake, the prior art includes reports of compounds and compositions that act as agonists or antagonists of MC4-R. As examples, U.S. Pat. No. 6,060,589 describes polypeptides that are capable of modulating signaling activity of melanocortin receptors. Also, U.S. Pat. Nos. 6,054,556 and 5,731,408 describe families of agonists and antagonists for MC4-R receptors that are lactam heptapeptides having a cyclic structure. WO 01/10842 discloses MC4-R binding compounds having a multitude of structures and methods of using such compounds to treat MC4-R associated disorders. Some of the compounds described include amidino- and guanidino-containing arenes and heteroarenes.

Various other classes of compounds have been disclosed as having MC4-R agonist activity. For example, WO 01/70708 and WO 00/74679 disclose MC4-R agonists that are piperidine compounds and derivatives, while WO 01/70337 and WO 99/64002 disclose MC-R agonists that are spiropiperidine derivatives. Other known melanocortin receptor agonists include aromatic amine compounds containing amino acid residues, particularly tryptophan residues, as disclosed in WO 01/55106. Similar agonists are disclosed in WO 01/055107 which comprise aromatic amine compounds containing tertiary amide or tertiary amine groups. Finally, WO 01/055109 discloses melanocortin receptor agonists comprising aromatic amines which are generally bisamides separated by a nitrogen-containing alkyl linker.

Guanidine-containing compounds having a variety of biological activities are also known in the prior art. For example, U.S. Pat. No. 4,732,916 issued to Satoh et al. discloses guanidine compounds useful as antiulcer agents; U.S. Pat. No. 4,874,864, U.S. Pat. No. 4,949,891, and U.S. Pat. No. 4,948,901 issued to Schnur et al. and EP 0343 894 disclose guanidino compounds useful as protease inhibitors and as anti-plasmin and anti-thrombin agents; and U.S. Pat. No. 5,352,704 issued to Okuyama et al. discloses a guanidino compound useful as an antiviral agent. Guanidine-containing compounds are also disclosed in other references. For example, U.S. Pat. No. 6,030,985 issued to Gentile et al. discloses guanidine compounds useful for treating and preventing conditions in which inhibition of nitric oxide synthetase is beneficial such as stroke, schizophrenia, anxiety, and pain. U.S. Pat. No. 5,952,381 issued to Chen et al. discloses certain guanidine compounds for use in selectively inhibiting or antagonizing αᵥβ₃ integrins.

Various 5-, 6-, and 7-membered fully saturated 1-azacarbocyclic-2-ylidene derivatives of guanidine are disclosed as having anti-secretory and hypoglycemic activities by U.S. Pat. No. 4,211,867 issued to Rasmussen. Such compounds are also taught as useful for the treatment of cardiovascular disease. Other guanidine derivatives are disclosed by U.S. Pat. No. 5,885,985 issued to Macdonald et al. as useful in therapy to treat inflammation.

Nevertheless, there remains a need for potent and specific agonists of MC4-R that are low molecular weight small molecules. Methods of treating a melanocortin-4 receptor mediated disease, such as obesity, with such non-peptide drugs, are,also particularly desirable.

### SUMMARY OF THE INVENTION

The instant invention provides potent and specific agonists of MC4-R that are low molecular weight small molecules. Thus, there has been provided, in accordance with one aspect of the invention, compounds of formula A¹-A²-A³-A⁴:

wherein

A¹ is a group of formula IIA or IIB;

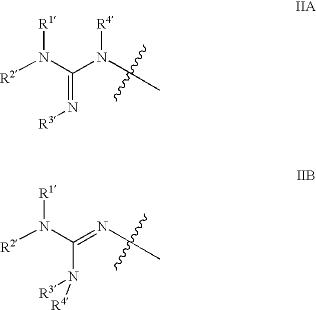

R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;

R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;

or R^{1′} and R^{2′}, together with the nitrogen to which they are bound, form a substituted or unsubstituted heterocyclyl or heteroaryl group;

R^{3′} is selected from the group consisting of substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;

R^{4′} is selected from the group consisting of H, and substituted and unsubstituted alkyl, alkenyl, alkynyl, cycloalkyl, heterocyclylalkyl, cycloalkylalkyl, aryl, heteroaryl, heterocyclyl, arylalkyl, and heteroarylalkyl groups;

A² is selected from the group consisting of substituted and unsubstituted aryl groups and substituted and unsubstituted heteroaryl groups;

A³ is a covalent bond such that A² is directly bonded to A⁴, or A³ is a linking group selected from the group consisting of O, S, —NR^{a}—, —C(═O)—, —C(═O)O—, —NR^{a}C(═O)—, —SO₂NR^{a}—, —C(═S)—, —C(═O)S—, —P(═O)R^{b}—, —SO₂—, and —S(═O)—, wherein if A³ is a linking group, then it is bonded to A² and A⁴ in a configuration selected from the group consisting of A²-O-A⁴, A²-S-A⁴, A²-NR^{a}-A⁴, A²-C(═O)-A⁴, A²-C(═O)O-A⁴, A⁴-C(═O)O-A², A²-NR^{a}C(═O)-A⁴, A⁴-NR^{a}C(═O)-A², A²-SO₂NR^{a}-A⁴, A⁴-SO₂NR^{a}-A², A²-C(═S)-A⁴, A²-(C═O)S-A⁴, A⁴-(C═O)S-A², A²-(P═O)R^{b}-A⁴, A²-SO₂-A⁴, and A²-S(═O)-A⁴ provided that if A³ is a linking group with the configuration A⁴-NR^{a}C(═O)-A², then A² is not a substituted or unsubstituted phenyl group and is not a substituted or unsubstituted 6-membered N-containing heteroaryl group;

A⁴ is selected from the group consisting of substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups;

R^{a} is selected from the group consisting of H, and substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups; and

R^{b} is selected from the group consisting of substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups.

Compounds provided by the invention further include prodrugs of the compound of A¹-A²-A³-A⁴, pharmaceutically acceptable salts thereof, stereoisomers thereof, tautomers thereof, hydrates thereof, hydrides thereof, or solvates thereof.

The invention provides further compounds of formula A¹-A²-A³-A⁴ in which A² is selected from the group consisting of substituted and unsubstituted phenyl groups and substituted and unsubstituted pyridyl groups.

The invention further provides compounds in which A³ is a linking group bonded to A² and A⁴ in a configuration selected from the group consisting of A²-NR^{a}-A⁴, A²-C(═O)-A⁴, A²-C(═O)O-A⁴, A⁴-C(═O)O-A², A²-NHC(═O)-A⁴, A²-SO₂NH-A⁴, and A²-SO₂-A⁴.

The invention provides further compounds of formula A¹-A²-A³-A⁴ in which R^{3′} is selected from the group consisting of substituted and unsubstituted cycloalkyl, polycyclic cycloalkyl, alkenyl, alkyl, and aryl groups. In other embodiments of compounds of formula A¹-A²-A³-A⁴, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments of compounds of formula A¹-A²-A³-A⁴, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, cyclohexylmethyl, isopinocampheyl, 7,7-dimethylnorbornyl, 4-isopropylcyclohexyl, and 3-methylcycloheptyl groups.

The invention provides further compounds of formula A¹-A²-A³-A⁴ in which R^{1′} is H and R^{2′} is selected from the group consisting of substituted and unsubstituted alkyl, arylalkyl, and heteroarylalkyl groups. In still other embodiments of compounds of formula A¹-A²-A³-A⁴, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of compounds of formula A¹-A²-A³-A⁴, R^{1′} and R^{2′} may be the same or different and are each independently selected from the group consisting of substituted and unsubstituted alkyl, arylalkyl, and heteroarylalkyl groups. In still other embodiments of compounds of formula A¹-A²-A³-A⁴, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups.

In still other embodiments of compounds of formula A¹-A²-A³-A⁴, R^{1′} and R^{2′}, together with the nitrogen to which they are bound, form a substituted or unsubstituted heterocyclyl group. In still other embodiments of compounds of formula A¹-A²-A³-A⁴, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In still other embodiments of compounds of formula A¹-A²-A³-A⁴, R^{1′} and R^{2′}, together with the nitrogen to which they are bound, form a substituted or unsubstituted piperazino, morpholino, pyrrolidino, piperidino, homopiperazino, or azepino group. In still other embodiments of compounds of formula A¹-A²-A³-A⁴, R^{1′} and R^{2′}, together with the nitrogen to which they are bound, form a piperazino group optionally substituted by one or two methyl groups.

The invention provides further compounds of formula A¹-A²-A³-A⁴ in which R^{a} is H.

The invention provides further compounds of formula A¹-A²-A³-A⁴ in which A³ is a covalent bond so that A² is directly bonded to A⁴.

The invention provides further compounds of formula A¹-A²-A³-A⁴ in which A⁴ is a 2,4-disubstituted phenylethyl group or an indolylethyl group. In still other embodiments of compounds of formula A¹-A²-A³-A⁴, A⁴ is selected from the group consisting of 2,4-dihalophenylethyl, and 2,4-dialkylphenylethyl groups. In still other embodiments of compounds of formula A¹-A²-A³-A⁴, A⁴ is selected from the group consisting of phenylethyl, 2,4-dichlorophenylethyl, 4-methoxyphenylethyl, 4-bromophenylethyl, 4-methylphenylethyl, 4-chlorophenylethyl, 4-ethylphenylethyl, cyclohexenylethyl, 2-methoxyphenylethyl, 2-chlorophenylethyl, 2-fluorophenylethyl, 3-methoxyphenylethyl, 3-fluorophenylethyl, thienylethyl, indolylethyl, 4-hydroxyphenylethyl, 3,4-dimethoxyphenylethyl, 2-chloro-4-iodophenylethyl, 2-fluoro-4-methylphenylethyl, 2-fluoro-4-bromophenylethyl, 2-fluoro-4-methoxyphenylethyl, 2-trifluoromethyl-4-fluorophenylethyl, 2,4-difluorophenylethyl, 2,4-dimethylphenylethyl, or 2,4-dimethoxyphenylethyl groups.

In accordance with another aspect of the invention, there has been provided, a compound of formula l:

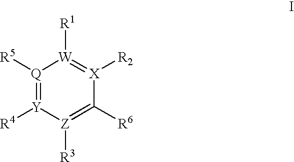

wherein

Q, W, X, Y, and Z are independently selected from the group consisting of carbon atoms and nitrogen atoms;

R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB;

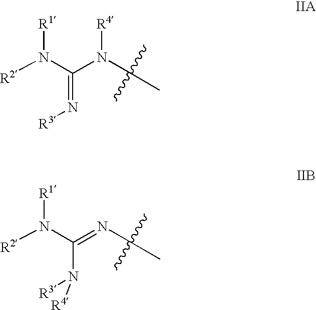

wherein R¹ may be absent if W is a nitrogen atom;

wherein R² may be absent if X is a nitrogen atom;

wherein R³ may be absent if Z is a nitrogen atom;

wherein R⁴ may be absent if Y is a nitrogen atom;

wherein R⁵ may be absent if Q is a nitrogen atom;

wherein one of R¹, R², R³, R⁴, or R⁵ is a group having the formula IIA or IIB;

R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;

R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;

or R^{1′} and R^{2′}, together with the nitrogen to which they are bound, form a substituted or unsubstituted heterocyclyl or heteroaryl group;

R^{3′} is selected from the group consisting of substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;

R^{4′} is selected from the group consisting of H, and substituted and unsubstituted alkyl, alkenyl, alkynyl, cycloalkyl, heterocyclylalkyl, cycloalkylalkyl, aryl, heteroaryl, heterocyclyl, arylalkyl, and heteroarylalkyl groups;

R⁶ is a group of formula IIIA, IIIB, IIIC, IIID, or IIIE;

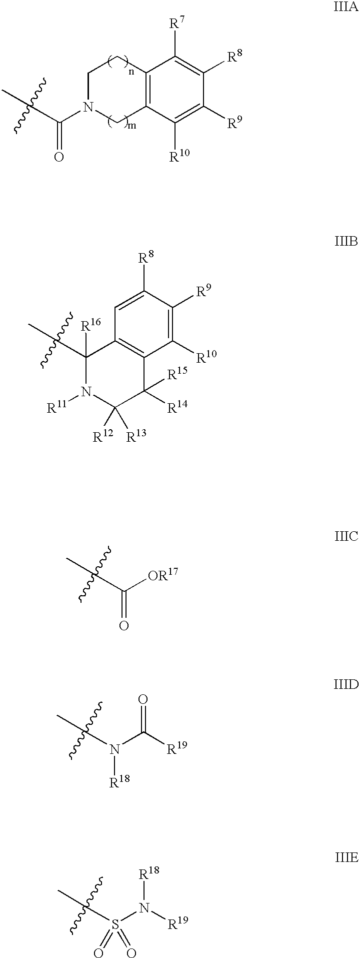

m is an integer selected from 0, 1, or 2;

n is an integer selected from 0, 1, or 2;

R⁷, R⁸, R⁹, and R¹⁰ may be the same or different and are independently selected from the group consisting of H, Cl, I, F, Br, OH, NH₂, CN, NO₂, and substituted and unsubstituted alkoxy, amino, alkyl, aryl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, and heteroarylaminocarbonyl groups;

R⁷ and R⁸ may join together with the carbon atoms to which they are attached to form a substituted or unsubstituted 5 or 6 membered ring;

R¹¹ is selected from the group consisting of H, and substituted and unsubstituted alkyl groups;

R¹², R¹³, R¹⁴, and R¹⁵ may be the same or different and are each independently selected from the group consisting of H, Cl, I, F, Br, OH, NH₂, CN, NO₂, and substituted and unsubstituted alkoxy, amino, alkyl, aryl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, and heteroarylaminocarbonyl groups;

R¹² and R¹⁴ may represent a second bond between the carbon bonded to R¹² and the carbon bonded to R¹⁴ such that the bond between the carbon bonded to R¹² and the carbon bonded to R¹⁴ is a double bond; and

R¹⁶ is selected from the group consisting of H, and substituted and unsubstituted alkyl groups;

R¹¹ and R¹⁶ may represent a second bond between the carbon bonded to R¹⁶ and the nitrogen bonded to R¹¹ such that the bond between the carbon bonded to R¹⁶ and the nitrogen bonded to R¹¹ is a double bond;

R¹⁷ is selected from the group consisting of H, and substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups;

R¹⁸ is selected from the group consisting of H, and substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups; and

R¹⁹ is selected from the group consisting of substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups.

Compounds provided by the invention further include prodrugs of the compound of formula I, pharmaceutically acceptable salts thereof, stereoisomers thereof, tautomers thereof, hydrates thereof, hydrides thereof, or solvates thereof.

In one embodiment R⁶ has the formula IIIA. In some embodiments where R⁶ has the formula IIIA, m is 0 and n is 2. In other embodiments where R⁶ has the formula IIIA, m is 1 and n is 1. In still other embodiments where R⁶ has the formula IIIA, m is 0 and n is 1. In yet other embodiments where R⁶ has the formula IIIA, m is 2 and n is 1.

In another embodiment R⁶ has the formula IIIB. In some embodiments where R⁶ has the formula IIIB, R¹¹ and R¹⁶ represent a second bond between the carbon bonded to R¹⁶ and the nitrogen bonded to R¹¹ such that the bond between the carbon bonded to R¹⁶ and the nitrogen bonded to R¹¹ is a double bond. In other embodiments where R⁶ has the formula IIIB, R¹¹ is H or a substituted or unsubstituted alkyl group and R¹⁶ is H.

In other embodiments in which R⁶ is a group of formula IIIA or IIIB, at least one of R⁸ or R⁹ is selected from the group consisting of Br, Cl, F, I substituted and unsubstituted alkyl groups, and substituted and unsubstituted alkoxy groups.

The invention provides further compounds of formula I in which R⁶ has the formula IIIC.

The invention provides further compounds of formula I in which R⁶ has the formula IIID. In other embodiments, R⁶ has the formula IIIE. In some embodiments in which R⁶ has the formula IIID or IIIE, R¹⁸ is H. In other embodiments in which R⁶ has the formula IIID, R¹⁹ is a substituted arylalkyl group, and the alkyl group of the R¹⁹ arylalkyl group is substituted with an amino or acetamido group.

In other embodiments in which R⁶ is a group of formula IIIC, IIID or IIIE, R¹⁷ or R¹⁹ is selected from the group consisting of substituted and unsubstituted arylalkyl groups, and substituted and unsubstituted heteroarylalkyl groups. In other embodiments in which R⁶ is a group of formula IIIC, IIID, or IIIE, R¹⁷ or R¹⁹ is a substituted or unsubstituted phenylalkyl group or a substituted or unsubstituted indolylalkyl group. In still other embodiments in which R⁶ is a group of formula IIIC, IIID, or IIIE, R¹⁷ or R¹⁹ is a 2,4-disubstituted phenylethyl group or an indolylethyl group. In still other embodiments in which R⁶ is a group of formula IIIC, IIID, or IIIE, R¹⁷ or R¹⁹ is selected from the group consisting of 2,4-dihalophenylethyl, and 2,4-dialkylphenylethyl groups. In still other embodiments in which R⁶ is a group of formula IIIC, IIID, or IIIE, R¹⁷ or R¹⁹ is selected from the group consisting of phenylethyl, 2,4-dichlorophenylethyl, 4-methoxyphenylethyl, 4-bromophenylethyl, 4-methylphenylethyl, 4-chlorophenylethyl, 4-ethylphenylethyl, cyclohexenylethyl, 2-methoxyphenylethyl, 2-chlorophenylethyl, 2-fluorophenylethyl, 3-methoxyphenylethyl, 3-fluorophenylethyl, thienylethyl, indolylethyl, 4-hydroxyphenylethyl, 3,4-dimethoxyphenylethyl, 2-chloro-4-iodophenylethyl, 2-fluoro-4-methylphenylethyl, 2-fluoro-4-bromophenylethyl, 2-fluoro-4-methoxyphenylethyl, 2-trifluoromethyl-4-fluorophenylethyl, 2,4-difluorophenylethyl, 2,4-dimethyl phenylethyl, or 2,4-dimethoxyphenylethyl groups. In some embodiments such as those described above in which R¹⁷ or R¹⁹ is a substituted or unsubstituted arylalkyl group such as a substituted or unsubstituted arylethyl group or more specifically a substituted phenylethyl group, the alkyl or ethyl group of the substituted or unsubstituted arylalkyl group is further substituted with a group such as an amino group; an alkylamino group such as a methylamino group; a hydroxyalkyl group such as a hydroxymethyl group; an —N(H)C(═O)-alkyl group such as a —N(H)C(═O)—CH₃ group; an —N(H)C(═O)—O-alkyl group such as a —N(H)C(═O)—O—C(CH₃)₃ group; or an —N(H)C(═O)—O-arylalkyl group such as a —N(H)C(═O)—O-benzyl group; an —N(H)C(═O)-heterocyclyl group such as a —N(H)C(═O)-(1,2,3,4-tetrahydroisoquinoline) group; or an arylalkoxyalkyl group, such as a phenylmethoxymethyl group, a 3-bromophenylmethoxymethyl group, a 4-methylphenylmethoxymethyl group, a 4-fluorophenylmethoxymethyl group, a 2-fluoro-4-chlorophenylmethoxymethyl group, and the like.

In other embodiments having any of the features described above, Q is a carbon atom and R⁵ is a group having the formula IIA or IIB.

In some embodiments, Q, W, X, Y, and Z are all carbon atoms whereas in other embodiments one of Q, W, X, Y, or Z is a nitrogen atom such that the ring containing Q, W, X, Y, and Z is a pyridine ring.

In other embodiments having any of the features described above, R^{4′} is H.

Other embodiments are provided which have any of the features described above in which R^{3′} is selected from the group consisting of substituted and unsubstituted cycloalkyl, polycyclic cycloalkyl, alkenyl, alkyl, and aryl groups. In still other embodiments, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, cyclohexylmethyl, isopinocampheyl, 7,7-dimethylnorbornyl, 4-isopropylcyclohexyl, and 3-methylcycloheptyl groups. In some embodiments, R^{3′} is a substituted cyclohexyl group such as a trifluoromethyl substituted cyclohexyl group such as a 4-trifluoromethylcyclohexyl group.

Other embodiments are provided which have any of the features described above in which R^{1′} is H and R^{2′} is selected from the group consisting of substituted and unsubstituted alkyl, arylalkyl, and heteroarylalkyl groups. In still other embodiments, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still further embodiments, R^{1′} and R^{2′} may be the same or different and are each independently selected from the group consisting of substituted and unsubstituted alkyl, arylalkyl, and heteroarylalkyl groups. In yet other embodiments, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments, R^{1′} and R^{2′}, together with the nitrogen to which they are bound, form a substituted or unsubstituted heterocyclyl group. In other embodiments, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In yet other embodiments, R^{1′} and R^{2′}, together with the nitrogen to which they are bound, form a substituted or unsubstituted piperazino, morpholino, pyrrolidino, piperidino, homopiperazino, or azepino group. In still further embodiments, R^{1′} and R^{2′}, together with the nitrogen to which they are bound, form a piperazino group optionally substituted by one or two methyl groups.

In some embodiments, if R¹⁷ is H or an unsubstituted alkyl group, then R^{1′} and R^{2′} join together, with the nitrogen atom to which they are bound, to form a substituted or unsubstituted heterocyclyl group. In some such embodiments, R^{3′} is a substituted cycloalkyl group or a substituted polycyclic cycloalkyl group. In other such embodiments, R^{1′} and R^{2′}, together with the nitrogen atom to which they are bound, form a substituted or unsubstituted heterocyclyl group that additionally includes an O, S, or an additional N atom. In some such embodiments R^{1′} and R^{2′}, together with the nitrogen atom to which they are bound, form a substituted or unsubstituted piperazino, morpholino, pyrrolidino, piperidino, homopiperazino, or azepino group.

There has also been provided, in accordance with another aspect of the invention, a composition comprising a compound according to the instant invention and a pharmaceutically acceptable carrier.

There has also been provided, in accordance with another aspect of the invention, a method of treating an MC4-R mediated disease, comprising administering to a subject in need thereof, a compound or composition of the instant invention.

In one embodiment, a disease to be treated by those methods of the instant invention is obesity or type II diabetes.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE PREFERRED EMBODIMENT

The instant invention relates to novel classes of small molecule melanocortin-4 receptor (MC4-R) agonists. These compounds can be formulated into compositions and are useful in activating MC4-R, or in the treatment of MC4-R-mediated diseases, such as obesity, type II diabetes, erectile dysfunction, polycystic ovary disease, complications resulting from or associated with obesity and diabetes, and Syndrome X.

The following definitions are used throughout this specification.

Alkyl groups include straight chain and branched alkyl groups having 1 to about 8 carbon atoms. Examples of straight chain alkyl groups include methyl, ethyl, propyl, butyl, pentyl, hexyl, heptyl, and octyl groups. Examples of branched alkyl groups, include, but not limited to, isopropyl, sec-butyl, t-butyl, and isopentyl groups. Representative substituted alkyl groups may be substituted one or more times with, for example, amino, thio, alkoxy, or halo groups such as F, Cl, Br, and I groups.

Cycloalkyl groups are cyclic alkyl groups such as, but not limited to, cyclopropyl, cyclobutyl, cyclopentyl, cyclohexyl, cycloheptyl, and cyclooctyl groups. Cycloalkyl groups also includes rings that are substituted with straight or branched chain alkyl groups as defined above, and further include cycloalkyl groups that are substituted with other rings including fused rings such as, but not limited to, decalinyl, tetrahydronaphthyl, and indanyl. Cycloalkyl groups also include polycyclic cycloalkyl groups such as, but not limited to, norbornyl, adamantyl, bornyl, camphenyl, isocamphenyl, and carenyl groups. Representative substituted cycloalkyl groups may be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which may be substituted with, for example, alkyl, alkoxy, amino, thio, or halo groups.

Alkenyl groups are straight chain, branched or cyclic lower alkyl groups having 2 to about 8 carbon atoms, and further including at least one double bond, as exemplified, for instance, by vinyl, propenyl, 2-butenyl, 3-butenyl, isobutenyl, cyclohexenyl, cyclopentenyl, cyclohexadienyl, butadienyl, pentadienyl, and hexadienyl groups among others.

Alkynyl groups are straight chain or branched lower alkyl groups having 2 to about 8 carbon atoms, and further including at least one triple bond, as exemplified by groups, including, but not limited to, ethynyl, propynyl, and butynyl groups.

Aryl groups are cyclic aromatic hydrocarbons that do not contain heteroatoms. Thus aryl groups include, but are not limited to, phenyl, azulene, heptalene, biphenylene, indacene, fluorene, phenanthrene, triphenylene, pyrene, naphthacene, chrysene, biphenyl, anthracenyl, and naphthenyl groups. Although the phrase “aryl groups” includes groups containing fused rings, such as fused aromatic-aliphatic ring systems, it does not include aryl groups that have other groups, such as alkyl or halo groups, bonded to one of the ring members. Rather, groups such as tolyl are referred to as substituted aryl groups. The phrase “aryl groups” includes groups bonded to one or more carbon atom(s), and/or nitrogen atom(s), in the compounds of formulas I and II. Representative substituted aryl groups may be mono-substituted or substituted more than once, such as, but not limited to, 2-, 3-, 4-, 5-, or 6-substituted phenyl or benzyl groups, which may be substituted with groups including, but not limited to, amino, alkoxy, alkyl, or halo.

Cycloalkylalkyl groups are alkyl groups as defined above in which a hydrogen or carbon bond of an alkyl group is replaced with a bond to a cycloalkyl group as defined above.

Arylalkyl groups are alkyl groups as defined above in which a hydrogen or carbon bond of an alkyl group is replaced with a bond to an aryl group as defined above.

Heterocyclyl groups are nonaromatic ring compounds containing 3 or more ring members, of which, one or more is a heteroatom such as, but not limited to, N, O, and S. The phrase “heterocyclyl group” includes fused ring species including those comprising fused aromatic and nonaromatic groups. The phrase also includes polycyclic ring systems containing a heteroatom such as, but not limited to quinuclidyl. However, the phrase does not include heterocyclyl groups that have other groups, such as alkyl or halo groups, bonded to one of the ring members. Rather, these are referred to as “substituted heterocyclyl groups”. Heterocyclyl groups include, but are not limited to, piperazino, morpholino, thiomorpholino, pyrrolidino, piperidino and homopiperazino groups. Representative substituted heterocyclyl groups may be mono-substituted or substituted more than once, such as, but not limited to morpholino or piperazino groups, which are 2-, 3-, 4-, 5-, or 6-substituted, or disubstituted with groups including, but not limited to, amino, alkoxy, alkyl, or halo.

Heteroaryl groups are aromatic ring compounds containing 3 or more ring members, of which, one or more is a heteroatom such as, but not limited to, N, O, and S. Heteroaryl groups include, but are not limited to, groups such as furan, thiophene, pyrrole, isopyrrole, diazole, imidazole, isoimidazole, triazole, dithiole, oxathiole, isoxazole, oxazole, thiazole, isothiazole, oxadiazole, oxatriazole, dioxazole, oxathiazole, pyran, dioxin, pyridine, pyrimidine, pyridazine, pyrazine, triazine, oxazine, isoxazine, oxathiazine, azepin, oxepin, thiepin, diazepine, benzofuran, and isobenzofuran. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups”. Representative substituted heteroaryl groups may be substituted one or more times with groups including, but not limited to, amino, alkoxy, alkyl, or halo.

Heterocyclylalkyl groups are alkyl groups as defined above in which a hydrogen or carbon bond of an alkyl group is replaced with a bond to a heterocyclyl group as defined above.

Heteroarylalkyl groups are alkyl groups as defined above in which a hydrogen or carbon bond of an alkyl group is replaced with a bond to a heteroaryl group as defined above.

Aminocarbonyl groups are groups of the formula RR′NC(O)—, wherein R or R′ may be the same or different, and each is independently selected from H, or substituted or unsubstituted alkyl, cycloalkyl, aryl, heterocyclyl or heteroaryl groups, as defined above.

In general, “substituted” refers to a group as defined above in which one or more bonds to a hydrogen atom contained therein are replaced by a bond to non-hydrogen or non-carbon atoms such as, but not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups and also substituted cycloalkyl groups also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles.

Substituted cycloalkyl, substituted aryl, substituted heterocyclyl and substituted heteroaryl also include rings and fused ring systems in which a bond to a hydrogen atom is replaced with a bond to a carbon atom. Therefore, substituted cycloalkyl, substituted aryl, substituted heterocyclyl and substituted heteroaryl groups may be substituted with alkyl groups as defined above.

Pharmaceutically acceptable salts include a salt with an inorganic base, organic base, inorganic acid, organic acid, or basic or acidic amino acid. As salts of inorganic bases, the invention includes, for example, alkali metals such as sodium or potassium, alkali earth metals such as calcium and magnesium or aluminum, and ammonia. As salts of organic bases, the invention includes, for example, trimethylamine, triethylamine, pyridine, picoline, ethanolamine, diethanolamine, triethanolamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

Prodrugs, as used in the context of the instant invention, includes those derivatives of the instant compounds which undergo in vivo metabolic biotransformation, by enzymatic or nonenzymatic processes, such as hydrolysis, to form a compound of the invention. Prodrugs can be employed to improve pharmaceutical or biological properties, as for example solubility, melting point, stability and related physicochemical properties, absorption, pharmacodynamics and other delivery-related properties.

The instant invention provides potent and specific agonists of MC4-R that are low molecular weight small molecules. In accordance with one aspect of the invention, the invention provides compounds of A¹-A²-A³-A⁴. Compounds of the invention further include prodrugs of compounds of formula A¹-A²-A³-A⁴, pharmaceutically acceptable salts thereof, stereoisomers thereof, tautomers thereof, hydrates thereof, hydrides thereof, or solvates thereof.

In compounds of formula A¹-A²-A³-A⁴, A¹ is a group of formula IIA or IIB.

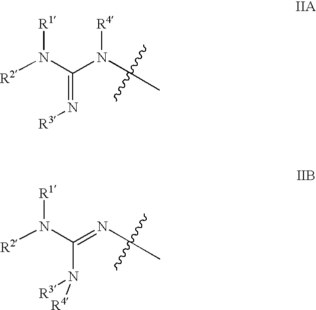

In compounds of formula A¹-A²-A³-A⁴, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In compounds of formula A¹-A²-A³-A⁴, R^{1′} and R^{2′}, together with the nitrogen atom to which they are both bound, may alternatively form a substituted or unsubstituted heterocyclyl or heteroaryl group. In one embodiment of compounds of formula A¹-A²-A³-A⁴, R^{1′} is H and R^{2′} is selected from the group consisting of substituted and unsubstituted alkyl, arylalkyl, and heteroarylalkyl groups. In still other embodiments of compounds of formula A¹-A²-A³-A⁴, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still further embodiments of compounds of formula A¹-A²-A³-A⁴, R^{1′} and R^{2′} may be the same or different and are each independently selected from the group consisting of substituted and unsubstituted alkyl, arylalkyl, and heteroarylalkyl groups. In yet other embodiments of compounds of formula A¹-A²-A³-A⁴, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of compounds of formula A¹-A²-A³-A⁴, R^{1′} and R^{2′}, together with the nitrogen to which they are bound, form a substituted or unsubstituted heterocyclyl group. In other embodiments of compounds of formula A¹-A²-A³-A⁴, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In another embodiment of compounds of formula A¹-A²-A³-A⁴, R^{1′} and R^{2′}, together with the nitrogen atom to which they are bound, form a substituted or unsubstituted heterocyclyl ring containing at least two nitrogen atoms. In still another embodiment of compounds of formula A¹-A²-A³-A⁴, R^{1′} and R^{2′}, together with the nitrogen atom to which they are bound, form a substituted or unsubstituted heterocyclyl ring containing at least one oxygen atom and one nitrogen atom. In yet other embodiments of compounds of formula A¹-A²-A³-A⁴, R^{1′} and R^{2′}, together with the nitrogen to which they are bound, form a substituted or unsubstituted piperazino, morpholino, pyrrolidino, piperidino, homopiperazino, or azepino group. In still further embodiments of compounds of formula A¹-A²-A³-A⁴, R^{1′} and R^{2′}, together with the nitrogen to which they are bound, form a piperazino group optionally substituted by one or two alkyl groups or in one embodiment by one or two methyl groups.

In compounds of formula A¹-A²-A³-A⁴, R^{3′} is selected from the group consisting of substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In one embodiment of compounds of formula A¹-A²-A³-A⁴, R^{3′} is selected from the group consisting of substituted and unsubstituted cycloalkyl, polycyclic cycloalkyl, alkenyl, alkyl, and aryl groups. In still other embodiments of compounds of formula A¹-A²-A³-A⁴, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments of compounds of formula A¹-A²-A³-A⁴, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, cyclohexylmethyl, isopinocampheyl, 7,7-dimethylnorbornyl, 4-isopropylcyclohexyl, and 3-methylcycloheptyl groups.

In compounds of formula A¹-A²-A³-A⁴, R^{4′} is selected from the group consisting of H, and substituted and unsubstituted alkyl, alkenyl, alkynyl, cycloalkyl, heterocyclylalkyl, cycloalkylalkyl, aryl, heteroaryl, heterocyclyl, arylalkyl, and heteroarylalkyl groups. In one embodiment of compounds of formula A¹-A²-A³-A⁴, R^{4′} is H.

In compounds of formula A¹-A²-A³-A⁴, A² is selected from the group consisting of substituted and unsubstituted aryl groups and substituted and unsubstituted heteroaryl groups. In one embodiment of compounds of formula A¹-A²-A³-A⁴, A² is selected from the group consisting of substituted and unsubstituted phenyl groups and substituted and unsubstituted pyridyl groups. In another embodiment, A² is a substituted or unsubstituted phenyl group and A¹ and A³ are ortho to one another on the A² phenyl group. In another embodiment, A² is a substituted or unsubstituted phenyl group and A¹ and A³ are para to one another on the A² phenyl group. In another embodiment, A² is a substituted or unsubstituted phenyl group and A¹ and A³ are meta to one another on the A² phenyl group. In another embodiment, A² is a substituted or unsubstituted phenyl group, A³ is a covalent bond, and A¹ and A⁴ are ortho to one another on the A² phenyl group. In another embodiment, A² is a substituted or unsubstituted phenyl group, A³ is a covalent bond, and A¹ and A⁴ are para to one another on the A² phenyl group. In yet another embodiment, A² is a substituted or unsubstituted phenyl group, A³ is a covalent bond, and A¹ and A⁴ are meta to one another on the A² phenyl group.

In compounds of formula A¹-A²-A³-A⁴, A³ is a covalent bond such that A² is directly bonded to A⁴. Alternatively, in compounds of formula A¹-A²-A³-A⁴, A³ is a linking group selected from the group consisting of O, S, —NR^{a}—, —C(═O)—, —C(═O)O—, —NR^{a}C(═O)—, —SO₂NR^{a}—, —C(═S)—, —C(═O)S—, —P(═O)R^{b}—, —SO₂—, and —S(═O)—. If A³ is a linking group, then it is bonded to A² and A⁴ in a configuration selected from the group consisting of A²-O-A⁴, A²-S-A⁴, A²-NR^{a}-A⁴, A²-C(═O)-A⁴, A²-C(═O))-A⁴, A⁴-C(═O)O-A², A²-NR^{a}C(═O)-A⁴, A⁴-NR^{a}C(═O)-A², A²-SO₂NR^{a}-A⁴, A⁴-SO₂NR^{a}-A², A²-C(═S)-A⁴, A²-(C═O)S-A⁴, A⁴-(C═O)S-A², A²-(P═O)R^{b}-A⁴, A²-SO₂-A⁴, and A²-S(═O)-A⁴. In compounds of formula A¹-A²-A³-A⁴, if A³ is a linking group with the configuration A⁴-NR^{a}C(═O)-A², then A² is not a substituted or unsubstituted phenyl group and is not a substituted or unsubstituted 6-membered N-containing heteroaryl group. In one embodiment of compounds of formula A¹-A²-A³-A⁴, A³ is a linking group such that A² is directly bonded to A⁴. In some embodiments, A³ is a linking group bonded to A² and A⁴ in a configuration selected from the group consisting of A²-NR^{a}-A⁴, A²-C(═O)-A⁴, A²-C(═O)O-A⁴, A⁴-C(═O)O-A², A²-NHC(═O)-A⁴, A²-SO₂NH-A⁴, and A²-SO₂-A⁴.

In compounds of formula A¹-A²-A³-A⁴, A⁴ is selected from the group consisting of substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups. In one embodiment of compounds of formula A¹-A²-A³-A⁴, A⁴ is a 2,4-disubstituted phenylethyl group or an indolylethyl group. In still other embodiments of compounds of formula A¹-A²-A³-A⁴, A⁴ is selected from the group consisting of 2,4-dihalophenylethyl, and 2,4-dialkylphenylethyl groups. In still other embodiments of compounds of formula A¹-A²-A³-A⁴, A⁴ is selected from the group consisting of phenylethyl, 2,4-dichlorophenylethyl, 4-methoxyphenylethyl, 4-bromophenylethyl, 4-methylphenylethyl, 4-chlorophenylethyl, 4-ethylphenylethyl, cyclohexenylethyl, 2-methoxyphenylethyl, 2-chlorophenylethyl, 2-fluorophenylethyl, 3-methoxyphenyl-ethyl, 3-fluorophenylethyl, thienylethyl, indolylethyl, 4-hydroxyphenylethyl, 3,4-dimethoxyphenylethyl, 2-chloro-4-iodophenylethyl, 2-fluoro-4-methylphenylethyl, 2-fluoro-4-bromophenylethyl, 2-fluoro-4-methoxyphenylethyl, 2-trifluoromethyl-4-fluorophenylethyl, 2,4-difluorophenylethyl, 2,4-dimethylphenylethyl, or 2,4-dimethoxyphenylethyl groups.

In some embodiments of compounds of formula A¹-A²-A³-A⁴, A⁴ is a substituted or unsubstituted arylalkyl or heteroarylalkyl group such as a substituted arylethyl, arylmethyl, heteroarylethyl, or heteroarylmethyl group, where the aryl or heteroaryl group is a group such as one of those included in Table I. In Table 1, Bn is benzyl, Cp is cyclopentyl, Pr is propyl, iPr is isopropyl, Et is ethyl, Me is methyl, Ph is phenyl, and t-Bu is t-butyl.

TABLE 1

| Aryl/Heteroaryl Group | R^{2′}, R^{b}, |
|---|---|
| 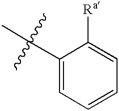 | R^{a′} = —H, —Cl, —F, —Br, —I, —CN, —Me, —Ph, —NHMe, —SH, —SMe, —OMe, —CH₂SO₂Ph, or —OCp |
| 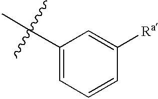 | R^{a′} = —F, —Cl, —Br, —I, —CN, —NO₂, —CN, —OMe, —Me, or —Ph |
| 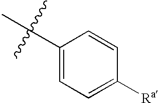 | R^{a′} = —F, —Cl, —Br, —I, —OMe, —OBn, —CF₃, —CN, —NO₂, —Me, —Ph, or —tBu |
| 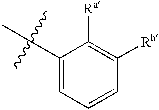 | R^{a′} = —OMe and R^{b′} =—OMe |
| 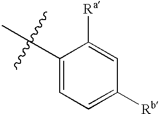 | R^{a′} = —OMe, —OEt, —OPr, —OiPr, —OCp, —CF₃, —Me, —Br, or —Cl; and R^{b′} = —OMe, —Me, —Ph, —tBu, —F, —Cl, —Br, —I, —NO₂, —CN, —CF₃, or —C(═O)CH₃ |
| 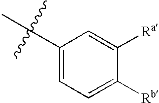 | R^{a′} = —Me or —Cl; and R^{b′} = —Me or —Cl |
| 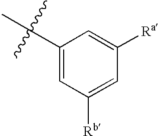 | R^{a′} = —CF₃ or —OMe; and R^{b′} —CF₃ or —OMe |
| 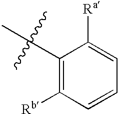 | R^{a′} = —F, —Cl, or —OMe; and R^{b′} —F, —Cl, OMe, or —Br |
| 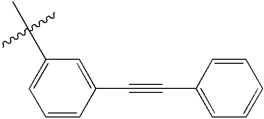 | Not Applicable |
| 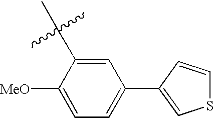 | Not Applicable |
| 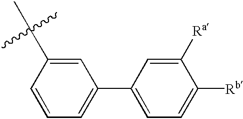 | R^{a′} = —H or —Cl; and R^{b′} =—OMe or —F |
| 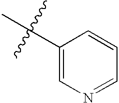 | Not Applicable |
| 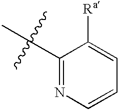 | R^{a′} = H or CF₃ |
| 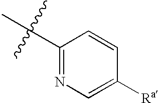 | R^{a′} = H or CF₃ |
| 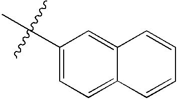 | Not Applicable |
| 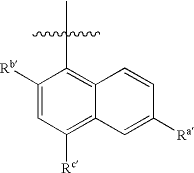 | R^{a′} = —H, —F, or —Br; R^{b′}= H, —OMe, —OEt, —OPr, —OiPr, or —OCp; and R^{c′} = —H, —F, or —Me |
| 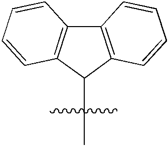 | Not Applicable |
| 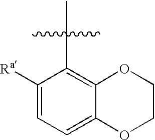 | R^{a′} = —H or —OMe |
| 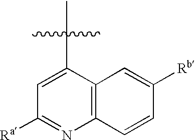 | R^{a′} = —H, —Cl, or —CF₃; and R^{b′} =—H or —Br |
| 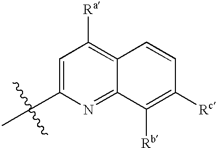 | R^{a′} = —H, or —Me; R^{b′} = —H or —Me; and R^{c′} = —H or —Cl |
| 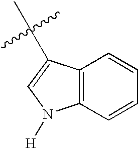 | Not applicable |
| 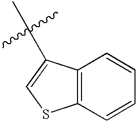 | Not applicable |
| 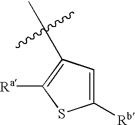 | R^{a′} = —H or —Cl; and R^{b′} = —H or —Cl |
| 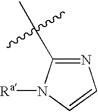 | R^{a′} = —H or —OBn |
| 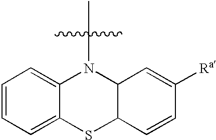 | R^{a′} = —H or —SMe |
| 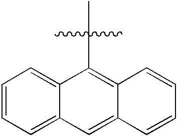 | Not applicable |

In compounds of formula A¹-A²-A³-A⁴, R^{a} is selected from the group consisting of H, and substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups. In one embodiment of compounds of formula A¹-A²-A³-A⁴, R^{a} is H.

In compounds of formula A¹-A²-A³-A⁴, R^{b} is selected from the group consisting of substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups.

In accordance with one aspect of the invention, the invention provides a first group of compounds of formula I such as shown below.

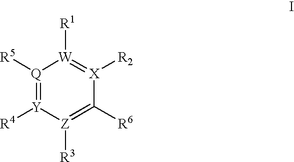

Compounds of the invention further include prodrugs of the first group of compounds of formula I, pharmaceutically acceptable salts thereof, stereoisomers thereof, tautomers thereof, hydrates thereof, hydrides thereof, or solvates thereof.

In the first group of compounds of formula I, Q, W, X, Y, and Z are independently selected from the group consisting of carbon atoms and nitrogen atoms. In some embodiments of the compounds of formula I, at least one of Q, W, X, Y, and Z is a nitrogen atom. In other embodiments of the compounds of formula I, Q, W, X, Y, and Z are all carbon atoms. In other embodiments of the compounds of formula I, Q is a nitrogen atom and W, X, Y, and Z are all carbon atoms. In other embodiments of the compounds of formula I, W is a nitrogen atom and Q, X, Y, and Z are all carbon atoms. In other embodiments of the compounds of formula I, X is a nitrogen atom and Q, W, Y, and Z are all carbon atoms. In still other embodiments of the compounds of formula I, Y is a nitrogen atom and Q, W, X, and Z are all carbon atoms. In still other embodiments of the compounds of formula I, Z is a nitrogen atom and Q, W, X, and Y are all carbon atoms.

In the first group of compounds of formula I, R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB.

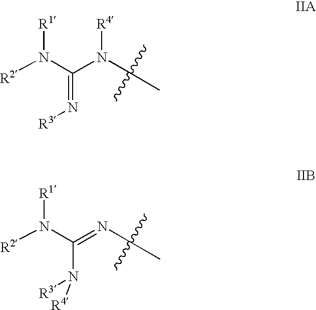

In the first group of compounds of formula I, R¹ may be absent if W is a nitrogen atom; R² may be absent if X is a nitrogen atom; R³ may be absent if Z is a nitrogen atom; R⁴ may be absent if Y is a nitrogen atom; and R⁵ may be absent if Q is a nitrogen atom. In the first group of compounds of formula I, at least one of R¹, R², R³, R⁴, or R⁵ is a group having the formula IIA or IIB. In some embodiments of the first group of compounds of formula I, Q is a carbon atom and R⁵ is a group having the formula IIA or IIB. In one embodiment, four of R¹ through R⁵ are H, and one of R¹ through R⁵is a group of formula IIA or IIB. In other embodiments, three of R¹ through R⁵ are H, one of R¹ through R⁵ is absent, one of R¹ through R⁵ is a group of formula IIA or IIB, one of W, Q, X, Y, and Z is a nitrogen atom, and four of W, Q, X, Y, and Z are carbon atoms.

In the first group of compounds of formula I, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In the first group of compounds of formula I, R^{1′} and R^{2′}, together with the nitrogen atom to which they are both bound, may alternatively form a substituted or unsubstituted heterocyclyl or heteroaryl group. In one embodiment R^{1′} is H and R^{2′} is selected from the group consisting of substituted and unsubstituted alkyl, arylalkyl, and heteroarylalkyl groups. In still other embodiments, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still further embodiments, R^{1′} and R^{2′} may be the same or different and are each independently selected from the group consisting of substituted and unsubstituted alkyl, arylalkyl, and heteroarylalkyl groups. In yet other embodiments, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments, R^{1′} and R^{2′}, together with the nitrogen to which they are bound, form a substituted or unsubstituted heterocyclyl group. In other embodiments, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In another embodiment, R^{1′} and R^{2′}, together with the nitrogen atom to which they are bound, form a substituted or unsubstituted heterocyclyl ring containing at least two nitrogen atoms. In still another embodiment, R^{1′} and R^{2′}, together with the nitrogen atom to which they are bound, form a substituted or unsubstituted heterocyclyl ring containing at least one oxygen atom and one nitrogen atom. In yet other embodiments, R^{1′} and R^{2′}, together with the nitrogen to which they are bound, form a substituted or unsubstituted piperazino, morpholino, pyrrolidino, piperidino, homopiperazino, or azepino group. In still further embodiments, R^{1′} and R^{2′}, together with the nitrogen to which they are bound, form a piperazino group optionally substituted by one or two alkyl groups or in one embodiment by one or two methyl groups.

In the first group of compounds of formula I, R^{3′} is selected from the group consisting of substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In one embodiment of the first group of compounds of formula I, R^{3′} is selected from the group consisting of substituted and unsubstituted cycloalkyl, polycyclic cycloalkyl, alkenyl, alkyl, and aryl groups. In still other embodiments, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, cyclohexylmethyl, isopinocampheyl, 7,7-dimethylnorbornyl, 4-isopropylcyclohexyl, and 3-methylcycloheptyl groups. In some embodiments, R^{3′} is a substituted cyclohexyl group such as a trifluoromethyl substituted cyclohexyl group such as a 4-trifluoromethylcyclohexyl group.

In the first group of compounds of formula I, R^{4′} is selected from the group consisting of H, and substituted and unsubstituted alkyl, alkenyl, alkynyl, cycloalkyl, heterocyclylalkyl, cycloalkylalkyl, aryl, heteroaryl, heterocyclyl, arylalkyl, and heteroarylalkyl groups. In one embodiment, R^{4′} is H.

In the first group of compounds of formula I, R⁶ is a group of formula IIIA, IIIB, IIIC, IIID, or IIIE.

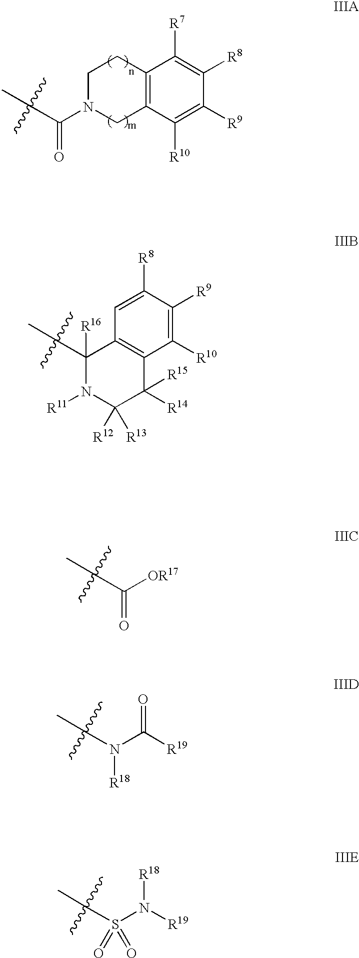

In some embodiments, R 6 has the formula IIIA. In other embodiments, R⁶ has the formula IIIB. In still other embodiments, R⁶ has the formula IIIC. In other embodiments, R⁶ has the formula IIID. In still other embodiments, R⁶ has the formula IIIE.

In the first group of compounds of formula I in which R⁶ is a group of formula IIIA, m is an integer selected from 0, 1, or 2, and n is an integer selected from 0, 1, or 2. In some embodiments where R⁶ has the formula IIIA, m is 0 and n is 2. In other embodiments where R⁶ has the formula IIIA, m is 1 and n is 1. In still other embodiments where R⁶ has the formula IIIA, m is 0 and n is 1. In yet other embodiments where R⁶ has the formula IIIA, m is 2 and n is 1. Examples of compounds in which m is 0 and n is 2, in which m is 1 and n is 1, in which m is 0 and n is 1, and in which m is 2 and n is 1 are respectively shown below as compounds of formula IVA, IVB, IVC, and IVD. In compounds of formula IVA through IVD, R¹ through R⁵, R⁷ through R⁹, Q, W, X, Y, Z, and R^{1′} through R^{4′} have the same definitions set forth elsewhere in this document.

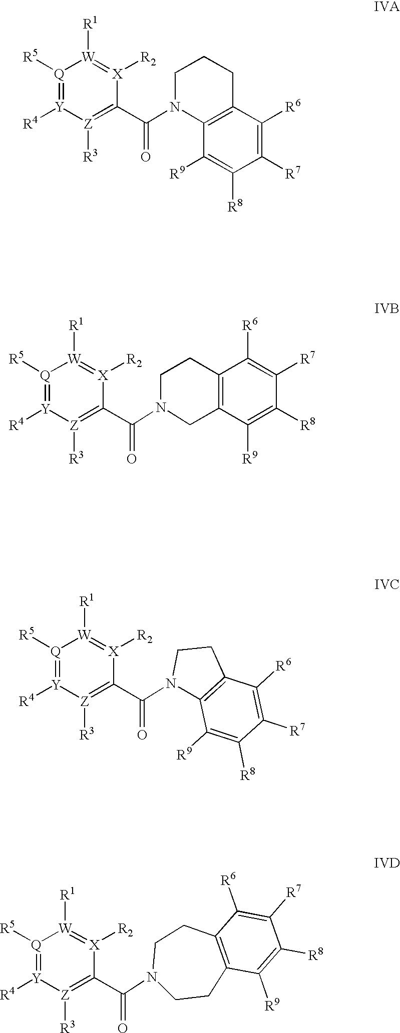

In the first group of compounds of formula I in which R⁶ is a group of formula IIIA or IIIB, R⁷, R⁸, R⁹, and R¹⁰ may be the same or different and are independently selected from the group consisting of H, Cl, I, F, Br, OH, NH₂, CN, NO₂, and substituted and unsubstituted alkoxy, amino, alkyl, aryl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, and heteroarylaminocarbonyl groups. In the first group of compounds of formula I in which R⁶ is a group of formula IIIA or IIIB, R⁷ and R⁸ may alternatively join together with the carbon atoms to which they are attached to form a substituted or unsubstituted 5 or 6 membered ring. In some embodiments in which R⁶ is a group of formula IIIA or IIIB, at least one of R⁸ or R⁹ is selected form the group consisting of Br, Cl, F, I, and substituted and unsubstituted alkyl groups, and alkoxy groups.

In the first group of compounds of formula I in which R⁶ is a group of formula IIIB, R¹¹ is selected from the group consisting of H, and substituted and unsubstituted alkyl groups.

In the first group of compounds of formula I in which R⁶ is a group of formula IIIB, R¹², R¹³, R¹⁴, and R¹⁵ may be the same or different and are independently selected from the group consisting of H, Cl, I, F, Br, OH, NH₂, CN, NO₂, and substituted and unsubstituted alkoxy, amino, alkyl, aryl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, and heteroarylaminocarbonyl groups. In the first group of compounds of formula I in which R⁶ is a group of formula IIIB, R¹⁶ is selected from the group consisting of H, and substituted and unsubstituted alkyl groups. In the first group of compounds of formula I in which R⁶ is a group of formula IIIB, R¹² and R¹⁴ may alternatively represent a second bond between the carbon bonded to R¹² and the carbon bonded to R¹⁴ such that the bond between the carbon bonded to R¹² and the carbon bonded to R¹⁴ is a double bond or is a bond of an aromatic ring. Such compounds have the formula VA. Furthermore, in the first group of compounds of formula I in which R⁶ is a group of formula IIIB, R¹¹ and R¹⁶ may represent a second bond between the carbon bonded to R¹⁶ and the nitrogen bonded to R¹¹ such that the bond between the carbon bonded to R¹⁶ and the nitrogen bonded to R¹¹ is a double bond or is a bond of an aromatic ring. Such compounds have the formula VB. In still other compounds of formula I, R¹² and R¹⁴ represent a second bond between the carbon bonded to R¹² and the carbon bonded to R¹⁴ such that the bond between the carbon bonded to R¹² and the carbon bonded to R¹⁴ is a double bond or is a bond of an aromatic ring, and R¹¹ and R¹⁶ represent a second bond between the carbon bonded to R¹⁶ and the nitrogen bonded to R¹¹ such that the bond between the carbon bonded to R¹⁶ and the nitrogen bonded to R¹¹ is a double bond or is a bond of an aromatic ring. Such compounds have the formula VC. In some embodiments in which R⁶ is a group of formula IIIB, R¹¹ is H or a substituted or unsubstituted alkyl group, and R¹⁶ is H. The variables in the compounds of formula VA, VB, and VC have the same definitions as described elsewhere in this document.

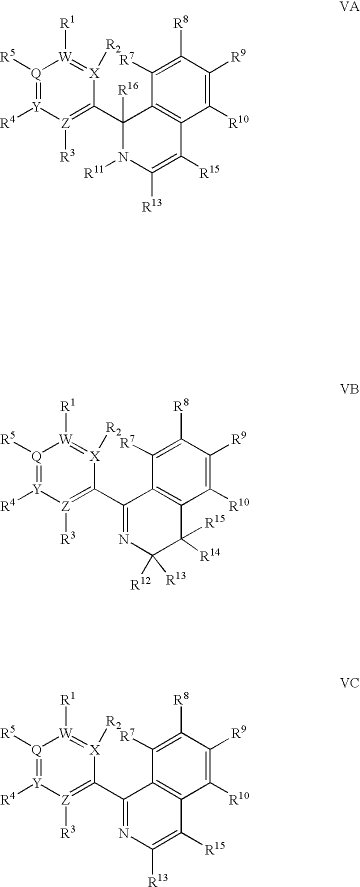

In the first group of compounds of formula I in which R⁶ is a group of formula IIIC, R¹⁷ is selected from the group consisting of H, and substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups. In some embodiments, if R¹⁷ is H or an unsubstituted alkyl group, then R^{1′} and R^{2′} join together, with the nitrogen atom to which they are bound, to form a substituted or unsubstituted heterocyclyl group. In some such embodiments, R^{3′} is a substituted cycloalkyl group or a substituted polycyclic cycloalkyl group. In other such embodiments, R^{1′} and R^{2′}, together with the nitrogen atom to which they are bound, form a substituted or unsubstituted heterocyclyl group that additionally includes an O, S, or an additional N atom. In some such embodiments R^{1′} and R^{2′}, together with the nitrogen atom to which they are bound, form a substituted or unsubstituted piperazino, morpholino, pyrrolidino, piperidino, homopiperazino, or azepino group.

In the first group of compounds of formula I in which R⁶ is a group of formula IIID or IIIE, R¹⁸ is selected from the group consisting of H, and substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups. In some embodiments in which R⁶ is a group of formula IIID, R¹⁸ is H. In some embodiments in which R⁶ is a group of formula IIIE, R¹⁸ is H.

In the first group of compounds of formula I in which R⁶ is a group of formula IIID or IIIE, R¹⁹ is selected from the group consisting of substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups. In some embodiments in which R⁶ is a group of formula IIID, R¹⁹ is a substituted arylalkyl group, and the alkyl group of the R¹⁹ arylalkyl group is substituted with an amino or acetamido group.

In some embodiments in which R⁶ is a group of formula IIIC, IIID, or IIIE, R¹⁷ or R¹⁹ is selected from the group consisting of substituted and unsubstituted arylalkyl, alkenyl, heteroarylalkyl, and heterocyclylalkyl groups. In other embodiments in which R⁶ is a group of formula IIIC, IIID, or IIIE, R¹⁷ or R¹⁹ is selected from the group consisting of substituted and unsubstituted arylalkyl groups, and substituted and unsubstituted heteroarylalkyl groups. In other embodiments in which R⁶ is a group of formula IIIC, IIID, or IIIE, R¹⁷ or R¹⁹ is a substituted or unsubstituted phenylalkyl group or a substituted or unsubstituted indolylalkyl group. In still other embodiments in which R⁶ is a group of formula IIIC, IIID, or IIIE, R¹⁷ or R¹⁹ is a 2,4-disubstituted phenylethyl group or an indolylethyl group. In still other embodiments in which R⁶ is a group of formula IIIC, IIID, or IIIE, R¹⁷ or R¹⁹ is selected from the group consisting of 2,4-dihalophenylethyl, and 2,4-dialkylphenylethyl groups. In still other embodiments in which R⁶ is a group of formula IIIC, IIID, or IIIE, R¹⁷ or R¹⁹ is selected from the group consisting of phenylethyl, 2,4-dichlorophenylethyl, 4-methoxyphenylethyl, 4-bromophenylethyl, 4-methylphenylethyl, 4-chlorophenylethyl, 4-ethylphenylethyl, cyclohexenylethyl, 2-methoxyphenylethyl, 2-chlorophenylethyl, 2-fluorophenylethyl, 3-methoxyphenylethyl, 3-fluorophenylethyl, thienylethyl, indolylethyl, 4-hydroxyphenylethyl, 3,4-dimethoxyphenylethyl, 2-chloro-4-iodophenylethyl, 2-fluoro-4-methylphenylethyl, 2-fluoro-4-bromophenylethyl, 2-fluoro-4-methoxyphenylethyl, 2-trifluoromethyl-4-fluorophenylethyl, 2,4-difluorophenylethyl, 2,4-dimethylphenylethyl, or 2,4-dimethoxyphenylethyl groups. In some embodiments such as those described above in which R¹⁷ or R¹⁹ is a substituted or unsubstituted arylalkyl group such as a substituted or unsubstituted arylethyl group or more specifically a substituted phenylethyl group, the alkyl or ethyl group of the substituted or unsubstituted arylalkyl group is further substituted with a group such as an amino group; an alkylamino group such as a methylamino group; a hydroxyalkyl group such as a hydroxymethyl group; an —N(H)C(═O)-alkyl group such as a —N(H)C(═O)—CH₃ group; an —N(H)C(═O)—O-alkyl group such as a —N(H)C(═O)—O—C(CH₃)₃ group; an —N(H)C(═O)—O-arylalkyl group such as a —N(H)C(═O)—O-benzyl group; an —N(H)C(═O)-heterocyclyl group such as a —N(H)C(═O)-(1,2,3,4-tetrahydroisoquinoline) group; or an arylalkoxyalkyl group, such as a phenylmethoxymethyl group, a 3-bromophenylmethoxymethyl group, a 4-methylphenylmethoxymethyl group, a 4-fluorophenylmethoxymethyl group, a 2-fluoro-4-chlorophenylmethoxymethyl group, and the like.

In some embodiments in which R⁶ is a group of formula IIIC, IIID, or IIIE, R¹⁷ or R¹⁹ is a substituted or unsubstituted arylalkyl or heteroarylalkyl group such as a substituted arylethyl, arylmethyl, heteroarylethyl, or heteroarylmethyl group, the aryl or heteroaryl group is a group such as one of those included in Table I above.

There has also been provided, in accordance with another aspect of the invention, a composition comprising a compound according to the instant invention and a pharmaceutically acceptable carrier.

There has also been provided, in accordance with another aspect of the invention, a method of activating MC4-R in a subject, comprising administering to a subject in need thereof an effective amount of a compound or composition of the instant invention.

There has also been provided, in accordance with another aspect of the invention, a method of treating an MC4-R-mediated disease, comprising administering to a subject in need thereof, a compound or composition of the instant invention.

In one embodiment, a disease to be treated by those methods of the instant invention is obesity, or type I or type II diabetes.

In another embodiment, a condition to be treated by those methods of the instant invention is a condition associated with or a complication arising from obesity or type II diabetes.

In another embodiment, a condition to be treated by those methods of the instant invention is erectile dysfunction.

In another embodiment, a disease to be treated by those methods of the instant invention is polycystic ovary disease.

In another embodiment, a disease to be treated by those methods of the instant invention is Syndrome X.

The invention also includes tautomers of the instant compounds. The instant invention also includes prodrugs, pharmaceutically acceptable salts, stereoisomers, hydrates, hydrides, and solvates of these tautomers.

The instant compounds may exist as one or more stereoisomers. The various stereoisomers include enantiomers, diastereomers, atropisomers and geometric isomers. In some cases, one stereoisomer may be more active and/or may exhibit beneficial effects in comparison to other stereoisomer(s) or when separated from the other stereoisomer(s). However, it is well within the skill of the ordinary artisan to separate, and/or to selectively prepare said stereoisomers. Accordingly, “stereoisomers” of the instant invention necessarily includes mixtures of stereoisomers, individual stereoisomers, or optically active forms.

The instant invention also provides for compositions which may be prepared by mixing one or more compounds of the instant invention, or pharmaceutically acceptable salts or tautomers thereof, with pharmaceutically acceptable carriers, excipients, binders, diluents or the like, to treat or ameliorate a variety of disorders. Examples of such disorders include, but are not limited to obesity, erectile disorders, cardiovascular disorders, neuronal injuries or disorders, inflammation, fever, cognitive disorders, sexual behavior disorders. A therapeutically effective dose further refers to that amount of one or more compounds of the instant invention sufficient to result in amelioration of symptoms of the disorder. The pharmaceutical compositions of the instant invention can be manufactured by methods well known in the art such as conventional granulating, mixing, dissolving, encapsulating, lyophilizing, emulsifying or levigating processes, among others. The compositions can be in the form of, for example, granules, powders, tablets, capsules, syrup, suppositories, injections, emulsions, elixirs, suspensions or solutions. The instant compositions can be formulated for various routes of administration, for example, by oral administration, by intranasal administration, by transmucosal administration, by rectal administration, or subcutaneous administration as well as intrathecal, intravenous, intramuscular, intraperitoneal, intranasal, intraocular or intraventricular injection. The compound or compounds of the instant invention can also be administered in a local rather than a systemic fashion, such as injection as a sustained release formulation. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive or excipient such as a starch or other additive. Suitable additives or excipients are sucrose, lactose, cellulose sugar, mannitol, maltitol, dextran, sorbitol, starch, agar, alginates, chitins, chitosans, pectins, tragacanth gum, gum arabic, gelatins, collagens, casein, albumin, synthetic or semi-synthetic polymers or glycerides, methyl cellulose, hydroxypropylmethyl-cellulose, and/or polyvinylpyrrolidone. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, a thickeners, buffers, a sweeteners, flavoring agents or perfuming agents. Additionally, dyestuffs or pigments may be added for identification. Tablets and pills may be further treated with suitable coating materials known in the art.

Liquid dosage forms for oral administration may be in the form of pharmaceutically acceptable emulsions, syrups, elixirs, suspensions, slurries and solutions, which may contain an inactive diluent, such as water. Pharmaceutical formulations may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For intranasal administration (e.g., to deliver compounds to the brain), or administration by inhalation (e.g., to deliver compounds through the lungs), the pharmaceutical formulations may be a solution, a spray, a dry powder, or aerosol containing any appropriate solvents and optionally other compounds such as, but not limited to, stabilizers, antimicrobial agents, antioxidants, pH modifiers, surfactants, bioavailability modifiers and combinations of these. Examples of intranasal formulations and methods of administration can be found in WO 01/41782, WO 00/33813, WO 91/97947, U.S. Pat. No. 6,180,603, and U.S. Pat. No. 5,624,898. A propellant for an aerosol formulation may include compressed air, nitrogen, carbon dioxide, or a hydrocarbon based low boiling solvent. The compound or compounds of the instant invention are conveniently delivered in the form of an aerosol spray presentation from a nebulizer or the like.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. The compounds may be formulated for parenteral administration by injection such as by bolus injection or continuous infusion. A unit dosage form for injection may be in ampoules or in multi-dose containers.

For rectal administration, the pharmaceutical formulations may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

The formulations of the invention may be designed for to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations may be compressed into pellets or cylinders and implanted intramuscularly or subcutaneously as depot injections or as implants such as stents. Such implants may employ known inert materials such as silicones and biodegradable polymers.

A therapeutically effective dose refers to that amount of the compound that results in amelioration of symptoms. Specific dosages may be adjusted depending on conditions of disease, the age, body weight, general health conditions, sex, diet of the subject, dose intervals, administration routes, excretion rate, and combinations of drugs. Any of the above dosage forms containing effective amounts are well within the bounds of routine experimentation and therefore, well within the scope of the instant invention. A therapeutically effective dose may vary depending upon the route of administration and dosage form. The preferred compound or compounds of the instant invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

The present invention also provides methods of enhancing MC4-R activity in a human or non-human animal. The method comprises administering an effective amount of a compound, or composition, of the instant invention to said mammal or non-human animal. Effective amounts of the compounds of the instant invention include those amounts that activate MC4-R which are detectable, for example, by an assay described below in the illustrative Examples, or any other assay known by those skilled in the art that a detect signal transduction, in a biochemical pathway, through activation of G-protein coupled receptors, for example, by measuring an elevated cAMP level as compared to a control model. Accordingly, “activating” means the ability of a compound to initiate a detectable signal. Effective amounts may also include those amounts which alleviate symptoms of a MC4-R disorder treatable by activating MC4-R.

An MC4-R disorder, or MC4-R-mediated disease, which may be treated by those methods provided, include any biological disorder or disease in which MC4-R is implicated, or which inhibition of MC4-R potentiates a biochemical pathway that is defective in the disorder or disease state. Examples of such diseases are obesity, erectile disorders, cardiovascular disorders, neuronal injuries or disorders, inflammation, fever, cognitive disorders, type II diabetes, polycystic ovary disease, Syndrome X, complications from obesity and diabetes, and sexual behavior disorders. In a preferred embodiment, the instant invention provides compounds, compositions, and methods effective for reducing energy intake and body weight; reducing serum insulin and glucose levels; alleviating insulin resistance; and reducing serum levels of free fatty acids. Accordingly, the instant invention is particularly effective in treating those disorders or diseases associated with obesity or type II diabetes.

“Treating” within the context of the instant invention, therefore, means an alleviation of symptoms associated with a disorder or disease, or halt of further progression or worsening of those symptoms, or prevention or prophylaxis of the disease or disorder. For example, within the context of obesity, successful treatment may include an alleviation of symptoms or halting the progression of the disease, as measured by reduction in body weight, or a reduction in amount of food or energy intake. In this same vein, successful treatment of type I or type II diabetes may include an alleviation of symptoms or halting the progression of the disease, as measured by a decrease in serum glucose or insulin levels in, for example, hyperinsulinemic or hyperglycemic patients.

The present invention, thus generally described, will be understood more readily by reference to the following examples, which are provided by way of illustration and are not intended to be limiting of the present invention.

### EXAMPLES

The following abbreviations are used throughout the Examples:

DIAD: Diisopropyl azodicarboxylate

DIEA: Diisopropylethylamine

DMF: Dimethylformamide

DMAP: 4-Dimethylaminopyridine

DMSO: Dimethylsulfoxide

EDCI: 1-Ethyl-3-(3′-dimethylaminopropyl)carbodiimide hydrochloride

HCl: Hydrochloric acid

KOH: Potassium hydroxide

LC: Liquid Chromatography

MS: Mass Spectroscopy

NaOH: Sodium Hydroxide

TFA: Trifluoroacetic acid

THF: Tetrahydrofuran

TLC: Thin Layer Chromatography

### Example 1

Step 1. General Synthesis of Aryl Azide and Nitroaryl Intermediates.

The 4-azido or 4-nitroarylcarboxylic acid starting materials in Examples A-D of Step 1 may also be functionalized as azido or nitropyridylcarboxylic acids. These are commercially available or may be prepared by the following known methods.

A. Carboxamides

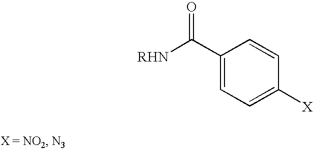

To a solution of an amine (1.0 equivalents) and 4-azido or4-nitroarylcarboxylic acid (1.0 equivalents) in THF was added EDCI (1.5 equivalents). The mixture was stirred at room temperature for 8-12 hours. THF was removed, and the residue was resuspended in ethyl acetate, washed with water, dried over sodium sulfate, concentrated, and purified by silica gel chromatography eluting with ethyl acetate/hexane or chloroform/methanol.

B. Amides

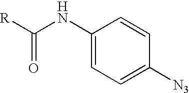

To a dry THF solution of an acid (0.5 M) and 4-azidoarylamine (1 equivalent) was added EDCI (1.5 equivalents). After stirring at room temperature for 8 hours, the reaction was concentrated in vacuo. The resulting mixture was diluted with ethyl acetate and washed with two portions of water. The organic layer was then isolated and dried over sodium sulfate. The solution was then filtered through a fritted funnel, concentrated, and dried overnight under high vacuum to yield the crude amidoarylazide product that was used without further purification.

C. Esters

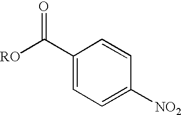

A dry THF solution containing a 4-nitroarylcarboxylic acid (1.5 equivalents), an alcohol (1.5 equivalents), DIAD (1.5 equivalents), and PPh₃ (1.5 equivalents) was refluxed. After stirring at reflux for 2 hours, the reaction was allowed to cool to room temperature and then concentrated in vacuo. The resulting mixture was dissolved in methylene chloride and purified via flash chromatography. The pure fractions were combined and concentrated in vacuo to yield the pure nitroester product.

D. Dihydroisoquinolines

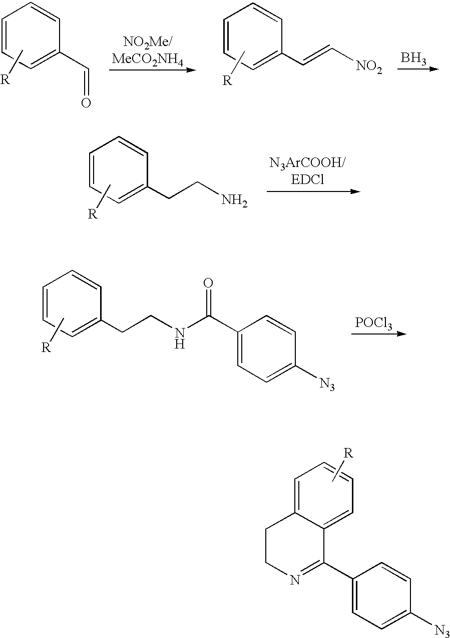

To an aryl aldehyde (1 equivalent) dissolved in acetic acid (0.66 M) was added nitromethane (3 equivalents) and ammonium acetate (1 equivalent) and the mixture was refluxed overnight. The reaction was cooled to room temperature and ethyl acetate was added. The organic phase was washed with water, NaHCO₃ (saturated aqueous), dried, and evaporated to yield a residue which was used without further purification.

The crude nitrostyrene product was dissolved in THF (0.2 M), was cooled to 0° C., and was treated with 1.0 M BH₃ in THF (5 equivalents). The reaction was then heated to reflux overnight. The reaction was cooled to 0° C. and quenched with H₂O and then 1N HCl was added until the pH was equal to about 2. The reaction was stirred for 30 minutes at room temperature and then extracted with ether (3×). The aqueous layer was made basic with 5% NaOH solution and then extracted into ether (3×). The combined organic layers were washed with brine, dried over Na₂SO₄, filtered, concentrated, and purified by silica gel chromatography. The amine was then coupled to a 4-azido or 4-nitroarylcarboxylic acid (EDCI, THF) as described in the carboxamide synthesis above.

The resulting carboxamide was suspended in POCl₃, and the mixture was heated at reflux for 1-3 days. The reaction was then cooled to room temperature and cautiously poured onto ice. The aqueous mixture was washed with chloroform, and the organic layer was washed with Na₂CO₃ (saturated aqueous). The acidic aqueous phase was cooled at 0° C. and made basic by addition of solid KOH. The resulting mixture was extracted with chloroform and the organic layers were combined, dried, and concentrated in vacuo. The resulting residue was purified on silica gel, eluting with chloroform/methanol.

E. Sulfonamides

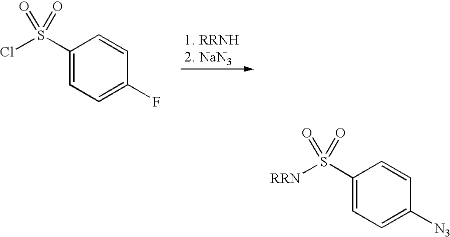

To a dry THF solution containing an amine (1.0 equivalent, 0.5 M in THF) and 4-fluorobenzenesulfonyl chloride (1.0 equivalent) was added ethyldiisopropylamine (1.1 equivalent). After stirring at room temperature for 12 hours, the reaction was concentrated in vacuo. The resulting mixture was diluted with ethyl acetate and washed with water (3×). The organic layer was then separated and dried over sodium sulfate. The solution was then filtered through a fritted funnel and concentrated to yield the crude product.

To a DMSO solution of the crude intermediate (1.0 equivalent, 0.5 M in DMSO) was added sodium azide (10 equivalents) and tetrabutylammonium chloride (2.3 equivalents). The reaction was fitted with a condenser and heated to 100° C. for 12 hours. The reaction was then cooled to room temperature, diluted with ethyl acetate, and washed with water (3×). The organic layer was next separated, dried over sodium sulfate, filtered through cotton, concentrated, and dissolved in a minimal amount of ethyl acetate. The crude mixture was purified via flash chromatography using hexanes/ethyl acetate. The pure fractions were combined, concentrated, and dried overnight under high vacuum to yield the azide product.

Step 2. General Synthesis of Guanidine Products from Aryl Carboxamide, Amide, Ester, and Dihydroisoquinoline Intermediates of Step 1.

A. Preparation from Aryl Azides

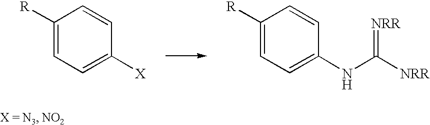

To a solution of the corresponding aryl azide (1.0 equivalent) in THF was added triphenylphosphine (1.0 equivalent) or tributylphosphine (1.0 equivalent, for use particularly with pyridylazide compounds) at room temperature. After 8 hours, the corresponding isocyanate was added (1.3 equivalents), and the solution was heated at 55-80° C. overnight. To the mixture was added an amine (1.3 equivalents). After being heated at the same temperature for 2 hours, THF was removed. The residue was resuspended in ethyl acetate, washed with water, dried over anhydrous sodium sulfate, concentrated, and purified by silica gel chromatography.

B. Preparation from Nitroaryl Compounds

A nitroaryl compound was taken up in ethanol (or methanol) and purged with dry nitrogen. To this solution was introduced activated Pd/C (10% w/w, 0.1 equivalent), and the mixture was hydrogenated for about 30 minutes or until complete by LC/MS. The mixture was then filtered through Celite, concentrated in vacuo, and taken on crude to the next step.

To a 0.5 M acetone solution (0° C. ice bath) containing the amine (1 equivalent) and sodium carbonate (3 equivalents) was added thiophosgene (3 equivalents) dropwise. After 2 hours at room temperature, the reaction mixture was concentrated in vacuo to remove solvent and excess thiophosgene. The residue was taken up in ethyl acetate and washed with water, dried with sodium sulfate, and then concentrated in vacuo to yield the isothiocyanate. To a solution of the resulting isothiocyanate in dry THF (0.5 M solution) was added an amine (1.5 equivalents). After stirring overnight, the reaction mixture was concentrated in vacuo and the thiourea product was dissolved in ethyl acetate or methylene chloride and purified via flash chromatography.

To a solution of the thiourea in dry THF (0.1 M) was added EDC (2 equivalents) and the solution heated at reflux (−80° C. external temp) for 60 minutes, after which it was cooled to room temperature and then placed in an ice bath for 15 minutes with stirring. A methylene chloride solution containing an amine (2 equivalents) was added and the reaction was stirred at room temperature. After 20 minutes, the reaction was diluted with ethyl acetate and washed with water. The aqueous layer was back extracted with ethyl acetate and the combined organic layers, after concentration in vacuo, was purified by silica gel flash chromatography.

### Example 2

The syntheses of additional starting materials that may be used in the general procedures of Example 1 are shown and described below.

A. Preparation of 1-(4-Azido-phenyl)-7-methoxy-2-methyl-1,2,3,4-tetrahydro-isoquinoline

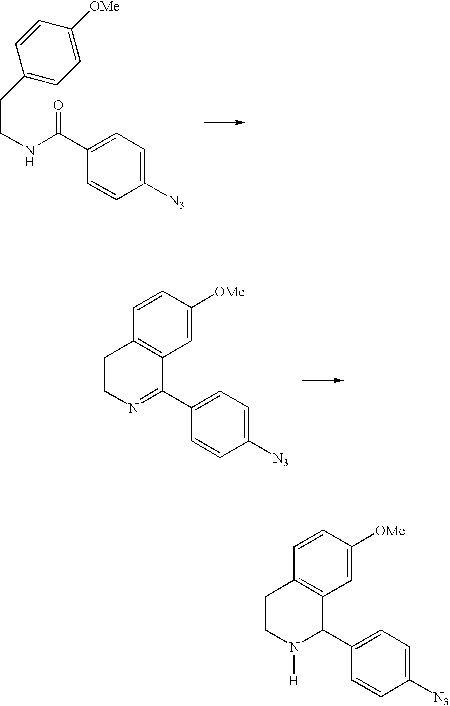

4-Azido-N-[2-(4-methoxy-phenyl)-ethyl]-benzamide was cyclized as described in Example 1, Step 1D. To the resulting 1-[4-(azadiazomvinyl)phenyl]-7-methoxy-3,4-dihydroisoquinoline (1 equivalent) in methanol was added paraformaldehyde (10 equivalents) and NaCNBH₃ (4 equivalents) and the reaction mixture was stirred at room temperature overnight. The reaction mixture was filtered through Celite, methanol was removed in vacuo, and the residue was dissolved in chloroform and washed with water. The organic extract was dried over magnesium sulfate and evaporated in vacuo to give the desired intermediate as an oil, which was used without further purification.

B. Preparation of 1-(4-Azido-phenyl)-7-methoxyisoquinoline

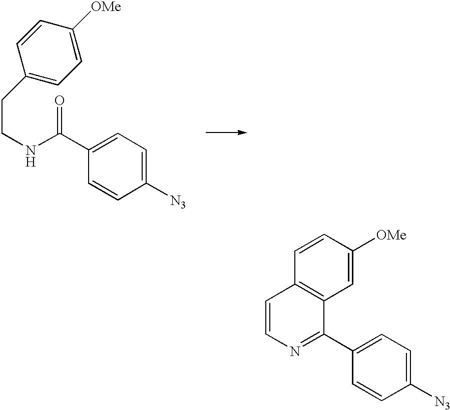

4-Azido-N-[2-(4-methoxy-phenyl)-ethyl]-benzamide was cyclized as described in Example 1, Step 1D. To a refluxing solution of 1-[4-(azadiazomvinyl)-phenyl]-7-methoxy-3,4-dihydroisoquinoline (1 equivalent) in dry benzene was added every hour activated MnO₂ (1.2 equivalent) (Dean-stark apparatus) for 8 hours, and the mixture was refluxed 24 hours. The reaction mixture was filtered through Celite, the filter cake washed with chloroform, and the filtrate evaporated in vacuo. The resulting crude product was purified on silica gel to separate the starting material, eluting with ethyl acetate/hexane 1:9 to 1:7.

C. Preparation of [1-(4-Azido-phenyl)-7-methoxy-3,4-dihydro-isoquinolin-3-yl]-methanol

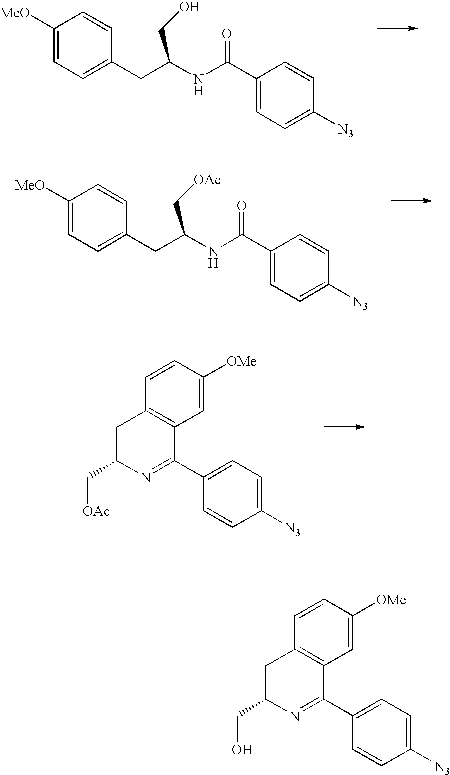

The hydroxymethyl carboxamide starting material was prepared from O-methyl-L-tyrosine following the procedure described in J. Org. Chem., 65, p. 503 (2000) and the coupling procedure in Example 1, Step 1A. A solution of the amide N-{(1S)-2-hydroxy-1-[(4-methoxyphenyl)methyl]ethyl}[4-(azadiazomvinyl)phenyl]-carboxamide (1 equivalent) in anhydrous pyridine and acetic anhydride (2 equivalents) was stirred at room temperature overnight. The reaction mixture was dissolved in ethyl acetate and washed with 1 M CuSO4. The organic extract was dried over magnesium sulfate and evaporated in vacuo to give a solid, which was used without further purification. The acetate was cyclized (POCl₃) as described in Example 1, step 1D. The cyclic acetate (1 equivalent) was dissolved in methanol and treated with K₂CO₃ (1 equivalent). After stirring at room temperature for 2 hours, the methanol was removed in vacuo, and the crude product was dissolved in chloroform and washed with water to yield [1-(4-Azido-phenyl)-7-methoxy-3,4-dihydro-isoquinolin-3-yl]-methanol.

D. Preparation of 2(S)-Amino-3-(2-fluoro4-methyl-phenyl)-propan-1-ol

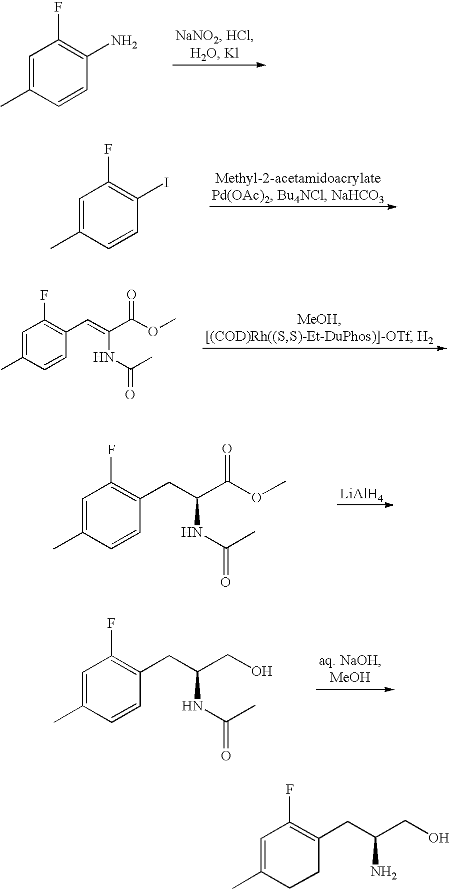

Synthesis of 2-Fluoro-1-iodo-methylbenzene

In a round bottom flask, 2-fluoro-4-methyl aniline (1 g, 7.99 mmol) was suspended in water (2 mL) and concentrated HCl (2 mL). This solution was then cooled in an ice bath with vigorous stirring. To this stirring solution was added sodium nitrite (662 mg, 9.58 mmol) dissolved in water (2 mL) dropwise over 30 minutes, keeping the temperature below 10° C. The reaction was then stirred for a further 30 minutes. The resulting solution was then added dropwise to a solution of potassium iodide (1.99 g, 11.98 mmol) dissolved in water (2 mL) stirring in an ice bath. The reaction was then refluxed for 2 hours before being allowed to stir at room temperature over night. The reaction was then taken up in ethyl acetate and washed with HCl (3 N), NaOH (1 M) containing a small portion of sodium metabisuffite. The organic layer was then dried over Na₂SO₄ and the solvent removed under reduced pressure to afford 1.46 g (77% yield) of a dark brown oil. This material was then purified via flash chromatography using a hexane running solvent and washed with 1 M HCl (2×), 2 M NaOH, brine and dried over Na₂SO₄ to recover the iodide product 829 mg (44% yield) of a colorless oil.

Synthesis of 2-Acetylamino-3-(2-fluoro-4-methyl-phenyl)-acrylic acid methyl ester via the Heck Reaction

A mixture of the aryl iodide (200 mg, 0.817 mmol), methyl-2-acetamidoacrylate (146 mg, 1.017 mmol), Pd(OAc)₂, (23 mg, 0.102 mmol), tetrabutylammonium chloride hydrate (283 mg, 1.017 mmol), and sodium hydrogen carbonate (192 mg, 2.288 mmol) was weighed into a 20 mL glass vial, flushed with nitrogen and sealed. The vial was then heated at 80° C. for 24 hours. The reaction was then cooled to room temperature and dissolved in methylene chloride. The organic layer was then washed with brine (3×) and dried over Na₂SO₄. The organic solvent was then removed under reduced pressure to yield a dark brown solid. This crude material was then purified via flash chromatography using 45% ethyl acetate/hexane running solvent to yield the Heck product 129 mg (60% yield) of an off white solid.

Hydrogenation to Form the 2(S)-Acetylamino-3-(2-fluoro-4-methyl-phenyl)-propionic acid methyl ester

In an oven dried Parr hydrogenation vial, the methyl ester above (129 mg, 0.51 mmol) was dissolved in anhydrous methanol (3.5 mL) along with the chiral catalyst (+)-1,2-Bis((2S,5S)-2,5-diethylphospholano)benzene (cyclooctadiene)rhodium(I)trifluoromethanesulfonate (4 mg, 5.5 μmol). The vial was then placed in the Parr pressure reactor, evacuated, and flushed with argon (5×) before evacuating and flooding with hydrogen (5×). The reaction was then allowed to proceed for 3 hours at room temperature with stirring. The reaction was then filtered through cotton wool before removing the organic solvent under reduced pressure to yield the product 100 mg (78% yield). This material was used without further purification. [M+H]+, 507.4.

Reduction of the Methyl Ester to Form the Acetamide

In an oven dried round bottom flask under nitrogen, LiAlH₄ (22 mg, 0.59 mmol) was suspended in anhydrous THF (2 mL) and cooled in an ice bath. To this stirring solution was added dropwise a THF (2 mL) solution of the product (50 mg, 0.19 mmol) from the previous step. The reaction was then allowed to warm to room temperature and monitored via TLC (45% ethyl acetate/hexane running solvent) until completion within approximately 1 hour. The reaction was then cooled in an ice bath and diluted with water and diethyl ether. To this vigorously stirring solution was added 2 M NaOH, and the reaction was then allowed to stir for a further 30 minutes. The aqueous layer was then extracted with diethyl ether (3×), and the combined organic extracts were dried over Na₂SO₄. The organic layer was then removed under reduced pressure to recover the product alcohol (35 mg, 79% yield) as an off white solid. [MH]+, 451.4.

Hydrolysis of the Acetamide to Form 2(S)-amino-3-(2-fluoro-4-methyl-phenyl)-propan-I-ol

In a round bottom flask, the acetamide from the previous step (4.748 g, 21.07 mmol) was dissolved in methanol (150 mL) and 2 M NaOH (150 mL) and refluxed. The reaction was then monitored via TLC using ethyl acetate and ninhydrin stain before it was allowed to cool to room temperature after 2 days. The reaction was then extracted with ethyl acetate (3×) and the organic layer dried over Na₂SO₄ before being removed under reduced pressure. This material was then purified via flash chromatography using a 10% methanol/methylene chloride/1% ammonia solution running solvent to give the amino alcohol 2.871 g (76% yield).

E. Preparation of 3-(2,4-Dimethylphenyl)-propionic acid

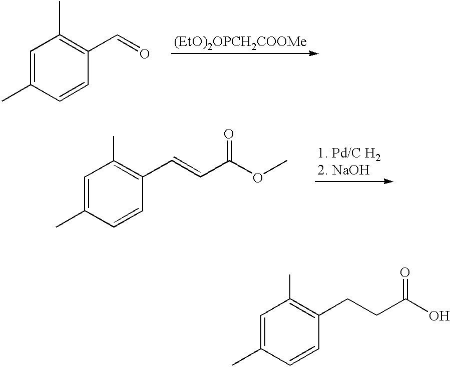

Synthesis of 3-(2,4-Dimethylphenyl)-acrylic acid methyl ester

In an oven dried round bottom flask under nitrogen, 2,4-dimethylbenzaldehyde (10 g, 74.52 mmol) and sodium hydride (3.28 g, 81.89 mmol) were suspended in anhydrous DMF (100 mL) and stirred in an ice bath. To this stirring solution was added dropwise methyl diethyl(phosphonoacetate) (15 mL, 81.98 mmol), and the solution was allowed to stir for a further 15 minutes before being allowed to warm to room temperature and proceed for two days. The reaction was then taken up in ethyl acetate and washed with 1 M HC1 (2×) and brine. The organic layer was then dried over sodium sulfate and the solvent removed under reduced pressure to recover the desired product 16.45 g. This material was used without further purification.

Hydrogenation to Yield 3-(2,4-Dimethylphenyl)-propionic acid methyl ester

3-(2,4-dimethylphenyl)-acrylic acid methyl ester (16.45 g, 86.44 mmol) was dissolved in methanol (120 mL) and evacuated (3×) connected to a hydrogenation apparatus. 10% Pd on C (1.0 g) was added to the flask under nitrogen, and the reaction was evacuated again. The vigorously stirring solution was then allowed to proceed under H₂ and monitored via NMR until the reaction was complete. After two days, the reaction was filtered through a Celite pad and concentrated to afford 3-(2,4-dimethylphenyl)-propionic acid methyl ester 15.1 g (90% yield). 193.1 [M+H]+.

Hydrolysis to yield 3-(2,4-Dimethylphenyl)-propionic acid

3-(2,4-dimethylphenyl)-propionic acid methyl ester (15.1 g, 78.54 mmol) was heated to reflux in 2.0 M NaOH (150 mL) overnight. The reaction was cooled, and washed with diethyl ether (2×) and the aqueous layer acidified with 2 N HCl to precipitate the desired 3-(2,4-dimethylphenyl)-propionic acid. The precipitate was collected by filtration and dried under vacuum (9.12 g, 68% yield).

### Example 3

Preparation of (3S)—N′-[4-(3,4-Dihydroquinolin-1(2H)-ylcarbonyl)phenyl]-3-methyl-N-[(1S,2S,3S,5R)-2,6,6-trimethylbicyclo[3.1.1]hept-3-yl]piperazine-1-carboximidamide

Step 1. Preparation of 1-[(4-Azidophenyl)carbonyl]-1,2,3,4-tetrahydroquinoline

A mixture of 1,2,3,4-tetrahydro-quinoline, 4-azidobenzoic acid, and 1-[3-(dimethlamino)propyl]-3-ethylcarbodiimide hydrochloride (1:1:1.5) were stirred in THF (0.43 M amine) for 20 hours at room temperature. After decanting and washing any remaining insoluble material with THF, the THF was removed in vacuo. The resulting solid was recrystallized from boiling ethyl acetate.

Step 2. Preparation of (3S)—N′-[4-(3,4-Dihydroquinolin-1(2H)-ylcarbonyl)phenyl]-3-methyl-N-[(1S,2S,3S,5R)-2,6,6-trimethylbicyclo[3.1.1]hept-3-yl]piperazine-1-carboximidamide

To a solution of 1-[(4-azidophenyl)carbonyl]-1,2,3,4-tetrahydroquinoline (1 equivalent; 0.1 M in anhydrous THF) was added trimethylphosphine (1 equivalent; 1 M in THF). After stirring for 10 minutes, (1S,2S,3S,5S)-2,6,6-trimethyl-bicyclo[3.1.1]heptan-3-isocyanate (1.3 equivalents) was added. After stirring at 55° C. for 18 hours, (S)-(+)-2-methylpiperazine (1.6 equivalents) was added, and the reaction was stirred at 55° C. for an additional 2 hours. Volatiles were removed in vacuo and the resulting off-white solid was run through a preparatory LC. Lyophilization of the pure fractions resulted in a fluffy white powder.

### Example 4

Preparation of (3S)-3-Methyl-N′-(4-{[7-(methyloxy)-3,4-dihydroisoquinolin-2(1H)-yl]carbonyl}phenyl)-N-[(1S,2S,3S,5R)-2,6,6-trimethylbicyclo[3.1.1]hept-3-yl]piperazine-1-carboximidamide

Step 1. Preparation of 4-Azido-N-{2-[4-(methyloxy)phenyl]ethyl}benzamide

A mixture of 2-(4-methoxy-phenyl)-ethylamine, 4-azidobenzoic acid, and 1-[3-(dimethlamino)propyl]-3-ethylcarbodiimide hydrochloride (1:1:1.5) were stirred in THF (0.43 M amine) for 20 hours at room temperature. After decanting and washing any remaining insoluble material with THF, the THF was removed in vacuo. The resulting solid was recrystallized from boiling ethyl acetate.

Step 2. Preparation of 2-[(4-Azidophenyl)carbonyl]-7-(methyloxy)-1,2,3,4-tetrahydroisoquinoline

To a mixture of 4-azido-N-{2-[4-(methyloxy)phenyl]ethyl}benzamide and paraformaldahyde (1:1.1) was added formic acid (0.35 M in benzamide). After stirring for 18 hours at 55° C., ethyl acetate was added, and the reaction was washed with saturated aqueous NaHCO₃ and saturated aqueous NaCl. The organic fraction was dried with MgSO₄, followed by removal of ethyl acetate in vacuo. Purification by flash chromatography, eluting with 30% ethyl acetate in hexanes resulted in a white solid.

Step 3. Preparation of (3S)-3-Methyl-N′-(4-{[7-(methyloxy)-3,4-dihydroisoquinolin-2(1H)-yl]carbonyl}phenyl)-N-[(1S,2S,3S,5R)-2,6,6-trimethylbicyclo[3.1.1]hept-3-yl]piperazine-1-carboximidamide

To a solution of 2-[(4-azidophenyl)carbonyl]-7-(methyloxy)-1,2,3,4-tetrahydroisoquinoline (1 equivalent; 0.1 M in anhydrous THF) was added trimethylphosphine (1 equivalent; 1 M in THF). After stirring for 10 minutes, (1S,2S,3S,5S)-2,6,6-trimethylbicyclo[3.1.1]heptan-3-isocyanate (1.3 equivalents) was added. After stirring at 55° C. for 18 hours, (S)-(+)-2-methylpiperazine (1.6 equivalents) was added, and the reaction was stirred at 55° C. for an additional 2 hours. Volatiles were removed in vacuo, and the resulting off-white solid was run through a preparatory LC. Lyophilization of the pure fractions resulted in a fluffy white powder.

### Examples 5-23

The compounds in the following table were prepared using the methodology described in Examples 3 and 4. The starting materials used in the syntheses are recognizable to one of skill in the art and are commercially available or may be prepared using known methods.

Table of Examples 5-23

| Example | Name | MH+ |
|---|---|---|
| 5 | (3S)-N′-{4-[(5,7-dimethyl-3,4-dihydroisoquinolin- | 542.8 |
| | 2(1H)-yl)carbonyl]phenyl}-3-methyl-N- | |
| | [(1S,2S,3S,5R)-2,6,6-trimethylbicyclo[3.1.1]hept-3- | |
| | yl]piperazine-1-carboximidamide | |
| 6 | (3S)-3-methyl-N′-(4-{[(6-(methyloxy)-3,4- | 544.7 |
| | dihydroisoquinolin-2(1H)-yl]carbonyl}phenyl)-N- | |
| | [(1S,2S,3S,5R)-2,6,6-trimethylbicyclo[3.1.1]hept-3- | |
| | yl]piperazine-1-carboximidamide | |
| 7 | (3S)-N-[4-(2,3-dihydro-1H-indol-1- | 500 |
| | ylcarbonyl)phenyl]-3-methyl-N′-[(1S,2S,3S,5R)- | |
| | 2,6,6-trimethylbicyclp[3.1.1]hept-3-yl]piperazine-1- | |
| | carboximidamide | |
| 8 | (3S)-N′-{5-[(7-bromo-3,4-dihydroisoquinolin-2(1H)- | 593.6 |
| | yl)carbonyl]pyridin-2-yl}-3-methyl-N-[(1S,2S,3S,5R)- | |
| | 2,6,6-trimethylbicyclo[3.1.1]hept-3-yl]piperazine-1- | |
| | carboximidamide | |
| 9 | (3S)-N′-{5-[(7-chloro-3,4-dihydroisoquinolin-2(1H)- | 549.2 |
| | yl)carbonyl]pyridin-2-yl}-3-methyl-N-[(1S,2S,3S5R)- | |
| | 2,6,6-trimethylbicyclo[3.1.1]hept-3-yl]piperazine-1- | |
| | carboximidamide | |
| 10 | (3S)-N′-{5-[(7-fluoro-3,4-dihydroisoquinolin-2(1H)- | 533.3 |
| | yl)carbonyl]pyridin-2-yl}-3-methyl-N-[(1S,2S,3S,5R)- | |
| | 2,6,6-trimethylbicyclo[3.1.1]hept-3-yl]piperazine-1- | |
| | carboximidamide | |
| 11 | (3S)-N-cycloheptyl-3-methyl-N′-(4-{[(7-(methyloxy)- | 504.2 |
| | 3,4-dihydroisoquinolin-2(1H)- | |
| | yl]carbonyl}phenyl)piperazine-1-carboximidamide | |
| 12 | (3S)-3-methyl-N-(4-methylcyclohexyl)-N′-(4-{[6- | 504.5 |
| | (methyloxy)-3,4-dihydroisoquinolin-2(1H)- | |
| | yl]carbonyl}phenyl)piperazine-1-carboximidamide | |
| 13 | (3S)-N′-(4-{[6,7-bis(methyloxy)-3,4- | 574.5 |
| | dihydroisoquinolin-2(1H)-yl]carbonyl}phenyl)-3- | |
| | methyl-N-[(1R,2S,3S,5S)-2,6,6- | |
| | trimethylbicyclo[3.1.1]hept-3-yl]piperazine-1- | |
| | carboximidamide | |
| 14 | (3S)-3-methyl-N-(4-methylcyclohexyl)-N′-(4-{[7- | 504.5 |
| | (methyloxy)-3,4-dihydroisoquinolin-2(1H)- | |
| | yl]carbonyl}phenyl)piperazine-1-carboximidamide | |
| 15 | (3S)-N-cycloheptyl-3-methyl-N′-(4-Δ[6-(methyloxy)- | 504.6 |
| | 3,4-dihydroisoquinolin-2(1H)- | |
| | yl]carbonyl}phenyl)piperazine-1-carboximidamide | |
| 16 | (3S)-N′-{4-[(5,7-dimethyl-3,4-dihydroisoquinolin- | 502.5 |
| | 2(1H)-yl)carbonyl]phenyl}-3-3methyl-N-(4- | |
| | methylcyclohexyl)piperazine-1-carboximidamide | |
| 17 | (3S)-N-cycloheptyl-N′-{4-[(5,7-dimethyl-3,4- | 502.5 |
| | dihydroisoquinolin-2(1H)-yl)carbonyl]phenyl}-3- | |
| | methylpiperazine-1-carboximidamide | |
| 18 | (3S)-N′-(4-{[6,7-bis(methyloxy)-3,4- | 534.5 |
| | dihydroisoquinolin-2(1H)-yl]carbonyl}phenyl)-N- | |
| | cycloheptyl-3-methylpiperazine-1-carboximidamide | |
| 19 | (3S)-N′-(4-{[6,7-bis(methyloxy)-3,4- | 534.3 |
| | dihydroisoquinolin-2(1H)-yl]carbonyl}phenyl)-3- | |
| | methyl-N-(4-methylcyclohexyl)piperazine-1- | |
| | carboximidamide | |
| 20 | (3S)-N-{4-[(7-bromo-3,4-dihydroisoquinolin-2(1H)- | 592.2 |
| | yl)carbonyl]phenyl}-3-methyl-N′-[(1S,2S,3R,5R)- | |
| | 2,6,6-trimethylbicyclo[3.1.1]hept-3-yl]piperazine-1- | |
| | carboximidamide | |
| 21 | (3S)-N-{4-[(7-chloro-3,4-dihydroisoquinolin-2(1H)- | 548.2 |
| | yl)carbonyl]phenyl}-3-methyl-N′-[(1S,2S,3R,5R)- | |
| | 2,6,6-trimethylbicyclo[3.1.1]hept-3-yl]piperazine-1- | |
| | carboximidamide | |
| 22 | (3S)-N-{4-[(7-fluoro-3,4-dihydroisoquinolin-2(1H)- | 532 |
| | yl)carbonyl]phenyl}-3-methyl-N′-[(1S,2S,5R)-2,6,6- | |
| | trimethylbicyclo[3.1.1]hept-3-yl]piperazine-1- | |
| | carboximidamide | |
| 23 | (3S)-3-methyl-N-(5-{]7-(methyloxy)-3,4- | 545 |
| | dihydroisoquinolin-2(1H)-yl]carbonyl}pyridin-2-yl)- | |
| | N′-[(1S,2S,3S,5R)-2,6,6-trimethylbicyclo[3.1.1]hept-3- | |
| | yl]piperazine-1-1carboximidamide | |

### Example 24

Preparation of (3S)-3-methyl-N-(4-{[7-(methyloxy)-1,2,4,5-tetrahydro-3H -3-benzazepin-3-yl]carbonyl}phenyl)-N′-[(1S,2S,3S,5R)-2,6,6-trimethylbicyclo[3.1.1]hept-3-yl]piperazine-1-carboximidamide

Step 1.

2-(3-methoxy-phenyl)-ethylamine (1 equivalent) was dissolved in anhydrous methylene chloride (0.88 M) in a three necked round bottom flask under N₂ and in an ice bath. Tosyl chloride (1.25 equivalent) was then dissolved in anhydrous methylene chloride under N₂ and added to this stirring solution over 10 minutes (Caution! This is an exothermic reaction). A precipitate formed, DIEA (1.2 equivalent) was then added, and the reaction was stirred at room temperature overnight. The reaction was then washed with 10% citric acid, 10% sodium carbonate, and brine before being dried over sodium sulfate. The organic solvent was then removed under reduced pressure to provide a brown oil. This crude material was then purified via flash chromatography using 100% methylene chloride running solvent to recover the product sulfonamide. (MH+) 306.1.

Step 2.

The sulfonamide (1 equivalent) produced in Step 1 was dissolved in acetone and stirred in a round bottom flask with K₂CO₃ (6.9 equivalents). The mixture was warmed to 78° C. and refluxed. Ethyl bromoacetate (1.5 equivalents) was then added, and the reaction was allowed to proceed overnight. The K₂CO₃ was then filtered off, and the solvent was removed under reduced pressure. To this colorless oil was added NaOH (4.4 equivalents) dissolved in 50% ethanol (0.4 M). The mixture was then warmed to reflux at 90° C. and allowed to proceed overnight. The ethanol was then removed under reduced pressure. The residual oil was then washed with water and extracted with diethyl ether. The aqueous layer was then acidified with concentrated HCl and extracted with diethyl ether (2×). The organic layers were then combined and extracted with sodium carbonate (2×). The aqueous layers were then combined and acidified with concentrated HCl and extracted with diethyl ether (2×). The organic layers were then combined and dried over sodium sulfate. The organic solvent was then removed under reduced pressure. The resulting material was then recrystallized from ethyl acetate/petroleum spirit to recover the alkylated product [[2-(3-methoxy-phenyl)-ethyl]-(toluene-4-sulfonyl)-amino]-acetic acid. (MH+) 363.9.

Step 3.

[[2-(3-Methoxy-phenyl )-ethyl]-(toluene-4-sulfonyl )-amino]-acetic acid (1 equivalent) was dissolved in anhydrous methylene chloride (0.13 M) and added to a stirring solution of P₂O₅ (5 equivalents) suspended in anhydrous methylene chloride (0.13 M) at 0° C. under nitrogen. The reaction was then allowed to proceed at room temperature for two days before being worked up. The reaction mixture was then diluted with 3% NaOH and extracted with methylene chloride. The organic layers were then combined and dried over sodium sulfate, and the solvent was removed under reduced pressure to recover the cyclized product 8-methoxy-3-(toluene-4-sulfonyl)-2,3,4,5-tetrahydro-benzo[d]azepin-1-one. The regio-isomer (ortho cyclized product) is formed in this reaction. The resulting material was purified via flash chromatography using 20% acetone/petroleum spirit running solvent. Two separate fractions of the desired isomeric pure 8-methoxy-3-(toluene-4-sulfonyl)-2,3,4,5-tetrahydro-benzo[d]azepin-1-one were recovered. These two fractions were treated separately for the next reaction. (MH+) 346.1.

Step 4.

The ketone product from Step 3 was dissolved in neat TFA and stirred under nitrogen. To this stirring solution was added triethyl silane (2.2 equivalents), and the reaction was allowed to proceed overnight at room temperature. Aqueous sodium carbonate was then added, and the solution was extracted with ether (2×). The ether layers were then combined and dried over sodium sulfate, and the solvent was removed under reduced pressure to recover an orange oil. The crude material from the two reactions was then combined and purified via flash chromatography using 20% acetone/1% ammonia solution/petroleum spirit to give 7-methoxy-3-(toluene-4-sulfonyl)-2,3,4,5-tetrahydro-1H-benzo[d]azepine. (MH+) 178.0.

Step 5.

Gaseous ammonia was first condensed into an oven dried three necked round bottom flask in a dry ice acetone bath under N₂. Sodium metal was then added to this vigorously stirring liquid ammonia to form sodium amide. The solution should hold a deep blue color to confirm that the liquid ammonia is anhydrous. The sulfonamide (1 equivalent) from Step 4 was then dissolved in THF (0.1 M) in an oven dried round bottom flask connected to a dry ice condenser. The anhydrous liquid ammonia was then distilled across into the round bottom flask containing the sulfonamide with vigorous stirring via the dry ice condenser connected in a series under a steady stream of N₂. Once the distillation had finished, the condenser and round bottom flask containing the sulfonamide was isolated. Sodium metal (2.1 equivalents) was then added until the solution again became a deep blue color. The reaction was stirred for a further 30 minutes before being quenched with NH₄Cl (9.3 equivalents). The reaction was then extracted with diethyl ether and dried over sodium sulfate, and the solvent was removed under reduced pressure to give the product amine as a yellow oil. (MH+) 353.3.

Step 6.

7-Methoxy-2,3,4,5-tetrahydro-1H-benzo[d]azepine (1 equivalent) was dissolved in THF (0.1 M) along with azidobenzoic acid (1.5 equivalents), EDCI (1.5 equivalents), DMAP (0.18 equivalents), and DIEA (1.5 equivalents). The reaction was stirred at room temperature overnight. The reaction was then washed with 10% citric acid, saturated sodium carbonate, and brine. The organic layer was then dried over sodium sulfate, and the organic solvent was removed under reduced pressure. The material was then purified via flash chromatography using 8% acetone/1% ammonia solution/petroleum spirit running solvent to give (4-azido-phenyl)-(7-methoxy-1,2,4,5-tetrahydro-benzo[d]azepin-3-yl)-methanone. (MH+) 323.2.

### Step 7.

(3S)-3-methyl-N-(4-{[7-(methyloxy)-1,2,4,5-tetrahydro-3H-3-benzazepin-3-yl]carbonyl}phenyl)-N′-[(1S,2S,3S,5R)-2,6,6-trimethyl-bicyclo[3.1.1]hept-3-yl]piperazine-1-carboximidamide was prepared using the product of Step 6 and following the procedure in Example 3. (MH+) 558.8.

### Examples 25-45

The compounds in the following table were prepared using the methodology described in Examples 1 and 2. The starting materials used in the syntheses are recognizable to one of skill in the art and are commercially available or may be prepared using known methods.

Table of Examples 25-45

| Example | Name | MH+ |
|---|---|---|
| 25 | (3S)-3-methyl-N-{4-[7-(methyloxy)-3,4- | 514 |
| | dihydroisoquinolin-1-yl]phenyl}-N′-[(1S,2S,3R,5R)- | |
| | 2,6,6-trimethylnicyclo[3.1.1]hept-3-yl]piperazine-1- | |
| | carboximidamide | |
| 26 | (3S)-3-methyl-N-[4-(7-methyl-3,4- | 498 |
| | dihydroisoquinolin-1-yl)phenyl]-N′-[(1S,2S,3R,5R)- | |
| | 2,6,6-trimethylbicyclo[3.1.1]hept-3-yl]piperazine-1- | |
| | carboximidamide | |
| 27 | (3S)-3-methyl-N-{4-[7-(methyloxy)-1,2,3,4- | 516 |
| | tetrahydroisoquinolin-1-yl]phenyl-N′- | |
| | [(1S,2S,3S,5R)-2,6,6-trimethylbicyclo[3.1.1]hept-3- | |
| | yl]piperazine-1-carboximidamide | |
| 28 | (3S)-N-{4-[6,7-bis(methyloxy)-3,4- | 544 |
| | dihydroisoquinolin-1-yl]phenyl}-3-methyl-N′- | |
| | [(1S,2S,3S,5R)-2,6,6-trimethylbicyclo[3.1.1]hept-3- | |
| | yl]piperazine-1-carboximidamide | |
| 29 | (3S)-3-methyl-N-{4-[6-(methyloxy)-3,4- | 514 |
| | dihydroisoquinolin-1-yl]phenyl}-N′-[(1S,2S,3R,5R)- | |
| | 2,6,6-trimethylbicyclo[3.1.1]hept-3-yl]piperazine-1- | |
| | carboximidamide | |
| 30 | (3S)-3-methyl-N-{4-[2-methyl-7-(methyloxy)-1,2,3,4- | 530 |
| | tetrahydroisoquinolin-1-yl]phenyl}-N′-[(1S,2S,3R)- | |
| | 2,6,6-trimethylbicyclo[3.1.1]hept-3-yl]piperazine-1- | |
| | carboximidamide | |
| 31 | (3S)-N-[4-(3,4-dihydrobenzo[h]isoquinolin-1- | 534 |
| | yl)phenyl]-3-methyl-N′-[(1S,2S,3R)-2,6,6- | |
| | trimethylbicyclo[3.1.1]jept-3-yl]piperazine-1- | |
| | carboximidamide | |
| 32 | (3S)-3-methyl-N-(4-{7-[(1-methylethyl)oxy]-3,4- | 542 |
| | dihydroisoquinolin-1-yl}phenyl)-N′-[(1S,2S,3R,5R)- | |
| | 2,6,6-trimethylbicyclo[3.1.1]hept-3-yl]piperazine-1- | |
| | carboximidamide | |
| 33 | (3S)-3-methyl-N-{4-[7-(methyloixy)isoquinolin-1- | 512 |
| | yl]phenyl}-N′-[(1S,2S,3R,5R)-2,6,6- | |
| | trimethylbicyclo[3.1.1]hept-3-yl]piperazine-1- | |
| | carboximidamide | |
| 34 | (3S)-3-methyl-N-{4-[7-(1-methylethyl)-3,4- | 526 |
| | dihydroisoquinolin-1-yl]phenyl}-N′-[(1S,2S,3R,5R)- | |
| | 2,6,6-trimethylbicyclo[3.1.]hept-3-yl]piperazine-1- | |
| | carboximidamide | |
| 35 | (3S)-N-{4-[7-(ethyloxy)-3,4-dihydroisoquinolin-1- | 528 |
| | yl]phenyl}-3-methyl-N′-[(1S,2S,3R,5R)-2,6,6- | |
| | trimethylbicyclo[3.1.1]hept-3-yl]piperazine-1- | |
| | carboximidamide | |
| 36 | (3S)-N-{4-[(3S)-3-(hydroxymethyl)-7-(methyloxy)- | 544 |
| | 3,4-dihydroisoquinolin-1-yl]phenyl}-3-methyl-N′- | |
| | [(1S,2S,3R,5R)-2,6,6-trimethylbicyclo[3.1.1]hept-3- | |
| | yl]piperazine-1-carboximidamide | |
| 37 | (3S)-3-methyl-N-{5-[7-(methyloxy)-3,4- | 515 |
| | dihydroisoquinolin-1-yl]pyridin-2-yl}-N′- | |
| | [(1S,2S,3R,5R)-2,6,6-trimethylbicyclo[3.1.1]hept-3- | |
| | yl]piperazine-1-carboximidamide | |
| 38 | (3S)-N-{5-[7-(ethyloxy)-3,4-dihydroisoquinolin-1- | 529 |
| | yl]pyridin-2-yl}-3-methyl-N′-[(1S,2S,3R,5R)-2,6,6- | |
| | trimethylbicyclo[3.1.1]hept-3-yl]piperazine-1- | |
| | carboximidamide | |
| 39 | (3S)-N-{4-[7-(butyloxy)-3,4-dihydroisoquinolin-1- | 556 |
| | yl]phenyl}-3-methyl-N′-[(1S,2S,3S,5R)-2,6,6- | |
| | trimethylbicyclo[3.1.1]hept-3-yl]piperazine-1- | |
| | carboximidamide | |
| 40 | (3R,5S)-N-{4-[7-(butyloxy)-3,4-dihydroisoquinolin-1- | 570 |
| | yl]phenyl}-3,5-dimethyl-N′-[(1S,2S,3S,5R)-2,6,6- | |
| | trimethylbicyclo[3.1.1]hept-3-yl]piperazine-1- | |
| | carboximidamide | |
| 41 | (3S)-N-{5-[7-(1,1-dimethylphenyl)-3,4- | 542 |
| | dihydroisoquinolin-1-yl]pyridin-2-yl}-3-methyl-N′- | |
| | [(1S,2S,3R,5R)-2,6,6-trimethylbicyclo[3.1.1]hept-3- | |
| | yl]piperazine-1-carboximidamide | |
| 42 | (3R,5S)-N-{5-[7-(ethyloxy)-3,4-dihydroisoquinolin-1- | 543 |
| | yl]pyridin-2-yl}-3,5-dimethyl-N′-[(1S,2S,3R,5R)- | |
| | 2,6,6-trimethylbicyclo[3.1.1]hept-3-yl]piperazine-1- | |
| | carboximidamide | |
| 43 | (3R,5S)-3,5-dimethyl-N-{5-[7-(1-methylethyl)-3,4- | 541 |
| | dihydroisoquinolin-1-yl]pyridin-2-yl}-N′- | |
| | [(1S,2S,3R,5R)-2,6,6-trimethylbicyclo[3.1.1]hept-3- | |
| | yl]piperazine-1-carboximidamide | |
| 44 | (3S)-N-{5-[7-(1,1-dimethylethyl)-3,4- | 541 |
| | dihydroisoquinolin-1-yl]pyridin-2-yl}-3-methyl-N′- | |
| | [(1S,2S,3R,5R)-2,6,6-trimethylbicyclo[3.1.1]hept-3- | |
| | yl]piperazine-1-carboximidamide | |
| 45 | (3S,5S)-N-{5-[7-(ethyloxy)-3,4-dihydroisoquinolin-1- | 544 |
| | yl]pyridin-2-yl}-3,5-dimethyl-N′-[(1S,2S,3S,5R)- | |
| | 2,6,6-trimethylbicyclo[3.1.1]hept-3-yl]piperazine-1- | |
| | carboximidamide | |

### Examples 46-76

The compounds in the following table were prepared using the methodology described in Examples 1 and 2. The starting materials used in the syntheses are recognizable to one of skill in the art and are commercially available or may be prepared using known methods.

Table of Examples 46-76

| Example | Name | MH+ |
|---|---|---|
| 46 | 2-(2,4-dichlorophenyl)ethyl 4-[((Z)-[(3S)-3- | 571.2 |
| | methylpiperazin-1-yl]{[(1S,2S,3S,5R)-2,6,6- | |
| | trimethylbicyclo[3.1.1]hept-3- | |
| | yl]imino}methyl)amino]benzoate | |
| 47 | 2-(2,4-dichlorophenyl)ethyl 4-[((Z)-[(3R,5S)-3,5- | 585.2 |
| | dimethylpiperazin-1-yl]{[(1S,2S,3S,5R)-2,6,6- | |
| | trimethylbicyclo[3.1.1]hept-3- | |
| | yl]imino}methyl)amino]benzoate | |
| 48 | 2-(2,4-dichlorophenyl)ethyl 4-[((Z)-[(3S)-3- | 585.1 |
| | methylpiperazin-1-yl]{[4- | |
| | (trifluoromethyl)cyclohexyl]imino}methyl)amino]ben- | |
| | zoate | |
| 49 | 4-[((Z)-[(3R,5S)-3,5-dimethylipierazin-1- | 413.2 |
| | yl]{[(1S,2S,3S,5R)-2,6,6-trimethylbicyclo[3.1.1]hept- | |
| | 3-yl]imino}methyl)amino]benzoic acid | |
| 50 | 4-chloro-N-{4-[((E)-[(3S)-3-methylpiperazin-1- | 551.3 |
| | yl]{[(1S,2S,3S,5R)-2,6,6-trimethylbicyclo[3.1.1]hept- | |
| | 3-yl]imino}methyl)amino]phenyl}-D- | |
| | phenylalaninamide | |
| 51 | ethyl 4-[((Z)-[(3R,5S)-3,5-dimethylpiperazin-1- | 441.3 |
| | yl]{[(1S,2S,3S,5R)-2,6,6-trimethylbicyclo[3.1.1]hept- | |
| | 3-yl]imino}methyl)amino]benzoate | |
| 52 | 3-[2-fluoro-4-(methyloxy)phenyl]-N-{4-[((Z)-[(3S)-3- | 550.3 |
| | methylpiperazin-1-yl]{[(1Sm2Sm3Sm5R)-2,6,6- | |
| | trimethylbicyclo[3.1.1]hept-3- | |
| | yl]imino}methyl)amino]phenyl}propanamide | |
| 53 | 3-(2,4-dimethylphenyl)-N-{4-[((Z)-[(3S)-3- | 530.3 |
| | methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 54 | 3-(2-fluoro-4-methylphenyl)-N-{4-[((Z)-[(3S)-3- | 534.3 |
| | methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 55 | 2,4-dichloro-N-{4-[((Z)-[(3S)-3-methylpiperazin-1- | 585.2 |
| | yl]{[(1S,2S,3S,5R)-2,6,6-trimethylbicyclo[3.1.1]hept- | |
| | 3-yl]imino}methyl)amino]phenyl}-L- | |
| | phenylalaninamide | |
| 56 | 2,4-dichloro-N-{4-[((Z)-[(3S)-3-methylpiperazin-1- | 585.2 |
| | yl]{[(1S,2S,3S,5R)-2,6,6-trimethylbicyclo[3.1.1]hept- | |
| | 3-yl]imino}methyl)amino]phenyl}-D- | |
| | phenylalaninamide | |
| 57 | 2,4-dichloro-N-{4-[((Z)-[(3R,5S)-3,5- | 599.3 |
| | dimethylpiperazin-1-yl]{](1S,2S,3S,5R)-2,6,6- | |
| | trimethylbicyclo[3.1.1]hept-3- | |
| | yl]imino}methyl)amino]phenyl}-D-phenylalaninamide | |
| 58 | N-{4-[((Z)-[(3R,5S)-3,5-dimethylpiperazin-1- | 546.3 |
| | yl]{[(1S,2S,3S,5R)-2,6,6-trimethylbicyclo[3.1.1]hept- | |
| | 3-yl]imino}methyl)amino]phenyl}-3-[4- | |
| | (methyloxy)phenyl]propanamide | |
| 59 | N-{4-[((Z)-[(3R,5S)-3,5-dimethylpiperazin-1- | 564.3 |
| | yl]{[(1S,2S,3S,5R)-2,6,6-trimethylbicyclo[3.1.1]hept- | |
| | 3-yl]imino}methyl)amino]phenyl}-3-[2-fluoro-4- | |
| | (methyloxy)propaninamide | |
| 60 | N-{4-[((Z)-[(3R,5S)-3,5-dimethylpiperazin-1- | 548.3 |
| | yl]{[(1S,2S,3S,5R)-2,6,6-trimethylbicyclo[3.1.1]hept- | |
| | 3-yl]imino}methyl)amino]phenyl}-3-(2-fluoro-4- | |
| | methylphenyl)propanamide | |
| 61 | 3-[2,4-bis(methyloxy)phenyl]-N-{4-[((Z)-[(3R,5S)- | 576.3 |
| | 3,5-dimethylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 62 | N-acetyl-N-{4-[((Z)-[(3R,5S)-3,5-dimethylpiperazin- | 605.4 |
| | 1-yl]{[(1S,2S,3S,5R)-2,6,6- | |
| | trimethylbicyclo[3.1.1]hept-3- | |
| | yl]imino}methyl)amino]phenyl}-2-fluoro-4-methyl-D- | |
| | phenylalaninamide | |
| 63 | N-acetyl-N-{4-[((Z)-[(3R,5S)-3,5-dimethylpiperazin- | 621.3 |
| | 1-yl]{[(1S,2S,3S,5R)-2,6,6- | |
| | trimethylbicyclo[3.1.1]hept-3- | |
| | yl]imino}methyl)amino]phenyl}-2-fluoro-O-methyl-D- | |
| | tyrosinamide | |
| 64 | N-acetyl-N-{4-[((Z)-[(3R,5S)-3,5-dimethylpiperazin- | 621.3 |
| | 1-yl]{[(1S,2S,3S,5R)-2,6,6-trimethyl- | |
| | bicyclo[3.1.1]hept-3-yl]imino}methyl)amino]phenyl}- | |
| | 2-fluoro-O-methyl-L-tyrosinamide | |
| 65 | N²-acetyl-3-(1,3-benzodioxol-4-yl)-N¹-{4-[((Z)- | 617.3 |
| | [(3R,5S)-3,5-dimethylpiperazin-1-yl]- | |
| | {[(1S,2S,3S,5R)-2,6,6-trimethylbicyclo[3.1.1]hept-3- | |
| | yl]imino}methyl)amino]phenyl}-D-alaninamide | |
| 66 | N-acetyl-N-{4-[((Z)-[(3R,5S)-3,5-dimethylpiperazin- | 609.3 |
| | 1-yl]{[(1S,2S,3S,5R)-2,6,6-trimethyl- | |
| | bicyclo[3.1.1]hept-3-yl]imino}methyl)amino]phenyl}- | |
| | 2,4-difluoro-D-phenylalaninamide | |
| 67 | N-{4-[((Z)-[(3R,5S)-3,5-dimethylpiperazin-1- | 603.3 |
| | yl]{[(1S,2S,3S,5R)-2,6,6-trimethylbicyclo[3.1.1]hept- | |
| | 3-yl]imino}methyl)amino]phenyl}-2-fluoro-O-methyl- | |
| | D-tyrosinamide | |
| 68 | N-{4-[((Z)-[(3R,5S)-3,5-dimethylpiperazin-1- | 563.4 |
| | yl]{[(1S,2S,3S,5R)-2,6,6-trimethylbicyclo[3.1.1]hept- | |
| | 3-yl]imino}methyl)amino]phenyl}-2-fluoro-4-methyl- | |
| | D-phenylalaninamide | |
| 69 | N-{4-[((Z)-[(3R,5S)-3,5-dimethylpiperazin-1- | 579.3 |
| | yl]{[(1S,2S,3S,5R)-2,6,6-trimethylbicyclo[3.1.1]hept- | |
| | 3-yl]imino}methyl)amino]phenyl}-2-fluoro-O-methyl- | |
| | D-tyrosinamide | |
| 70 | N-{4-[((Z)-[(3S,5R)-3,5-dimethylpiperazin-1- | 548.3 |
| | yl]{[(1S,2S,3S,5R)-2,6,6-trimethylbicyclo[3.1.1]hept- | |
| | 3-yl]imino}methyl)amino]phenyl}-3-(2-fluoro-4- | |
| | methylphenyl)propanamide | |
| 71 | N-{4-[((Z)-[(3S,5S)-3,5-dimethylpiperazin-1- | 564.3 |
| | yl]{[(1S,2S,3S,5R)-2,6,6-trimethylbicyclo[3.1.1]hept- | |
| | 3-yl]imino}methyl)amino]phenyl}-3-[2-fluoro-4- | |
| | (methyloxy)phenyl]propanamide | |
| 72 | (3S)-N-[4-({[2-(2,4- | 566.1 |
| | dichlorophenyl)ethyl]amino}sulfonyl)phenyl]-3- | |
| | methyl-N′-(2-methylcyclohexyl)piperazin-1- | |
| | carboximidamide | |
| 73 | (3S)-N′-cyclohexyl-N-[4-({[2-(2,4- | 552.1 |
| | dichlorophenyl)ethyl]amino}sulfonyl)phenyl]-3- | |
| | methylpiperazin-1-carboximidamide | |
| 74 | (3S)-N-[4-({[2-(2,4- | 566 |
| | dichlorophenyl)ethyl]amino}sulfonyl)phenyl]-3- | |
| | methyl-N′-(4-methylcyclohexyl)piperazine-1- | |
| | carboximidamide | |
| 75 | (3S)-N-[4-({[2-(2,4-dichlorophenyl)ethyl]amino}- | — |
| | sulfonyl)phenyl]-3-methyl-N-[(1S,2S,3S,5R)-2,6,6- | |
| | trimethylbicyclo[3.1.1]hept-3-yl]piperazine-1- | |
| | carboximidamide | |
| 76 | (3S)-N-[4-({[2-(2,4-dichlorophenyl)ethyl]amino}- | — |
| | sulfonyl)phenyl]-3-methyl-N′-spiro[2.5]oct-4- | |
| | ylpiperazine-1-carboximidamide | |

### Examples 77-135

The compounds in the following table were prepared using the methodology described in Examples 1 and 2. The starting materials used in the syntheses are recognizable to one of skill in the art and are commercially available or may be prepared using known methods. These compounds were named using using ACD name version 5.07 software (Nov. 14, 2001) available from Advanced Chemistry Development, Inc.

Table of Examples 77-135

| Example | Name | MH+ |
|---|---|---|
| 77 | (2S)-N-{4-[((Z)-[(3R,5S)-3,5-dimethylpiperazin-1- | 684.9 |
| | yl]{[(1S,2S,3S,5R)-2,6,6-trimethylbicyclo[3.1.1]hept- | |
| | 3-yl]imino}methyl)amino]phenyl}-3-[2-fluoro-4- | |
| | (methyloxy)phenyl]-2- | |
| | {[(phenylmethyl)oxy]methyl}propanamide | |
| 78 | (2R)-2-amino-3-(2,4-dichlorophenyl)-N-{4-[((Z)- | 586.6 |
| | [(3S)-3-methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 79 | (2S)-2-amino-3-(2,4-dichlorophenyl)-N-{4-[((Z)- | 586.6 |
| | [(3S)-3-methylpiperazin-1-yl]{[(1S,2S,3S,5R)-2,6,6- | |
| | trimethylbicyclo[3.1.1]hept-3- | |
| | yl]imino-56 methyl)amino]phenyl}propanamide | |
| 80 | 3-(4-bromo-2-fluorophenyl)-N-{4-[((Z)-[(3S)-3- | 599.6 |
| | methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 81 | (2R)-2-amino-3-(2,4-dichlorophenyl)-N-{4-[((Z)- | 586.6 |
| | [(3S)-3-methylpiperain-1-yl]{[(1S,2S,3S,5R)-2,6,6- | |
| | trimethylbicyclo[3.1.1]hept-3- | |
| | yl]imino}methyl)amino]phenyl}propanamide | |
| 82 | 3-[2,4-bis(methyloxy)phenyl]-N-{4-[((Z)-[(3S)-3- | 562.8 |
| | methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 83 | 1,1-dimethylethyl (1R)-1-[(4-chlorophenyl)methyl]-2- | 652.3 |
| | ({4-[((E)-[(3S)-3-methylpiperazin-1- | |
| | yl]{[(1S,2S,3S,5R)-2,6,6-trimethylbicyclo[3.1.1]hept- | |
| | 3-yl]imino}methyl)amino]phenyl}amino)-2- | |
| | oxoethylcarbamate | |
| 84 | 3-(4-bromo-2-fluorophenyl)-N-{4-[((E)-[(3R,5S)-3,5- | 613.6 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 85 | 3-(4-bromo-2-fluorophenyl)-N-{4-[((E)-[(3R,5S)-3,5- | 613.6 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 86 | (2R)-2-amino-3-(4-chlorophenyl)-N-{4-[((E)-[(3S)-3- | 552.2 |
| | methylpiperazin-1-yl]Δ[(1S,2S,3S,5R)-2,6,6- | |
| | trimethylbicyclo[3.1.1]hept-3- | |
| | yl]imino}methyl)amino]phenyl}propanamide | |
| 87 | (2R)-2-amino-3-(2,4-dichlorophenyl)-N-{4-[((Z)- | 586.6 |
| | [(3S)-3-methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 88 | 3-(2,4-dichlorophenyl)-N-{4-[((E)-[(3S)-3- | 571.6 |
| | methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 89 | 3-(2,4-dufluorophenyl)-N-{4-[((E)-[(3S)-3- | 538.7 |
| | methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 90 | 3-(2,4-dimethylphenyl)-N-{4-[((E)-[(3R,5S)-3,5- | 544.8 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 91 | 3-(2,4-dimethylphenyl)-N-{4-[((Z)-[(3S)-3- | 530.8 |
| | methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 92 | 3-(2,4-dichlorophenyl)-N-[4-({(E)-[(4- | 531.5 |
| | methylcyclohexyl)imino][(3S)-3-methylpiperazin-1- | |
| | yl]methyl}amino)phenyl]propanamide | |
| 93 | (2R)-2-amino-3-(2,4-dichlorophenyl)-N-{4-[((Z)- | 586.6 |
| | [(3S)-3-methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 94 | (2R)-2-amino-3-(2,4-dichlorophenyl)-N-{4-[((Z)- | 586.6 |
| | [(3S)-3-methylpiperazin-1-yl]{[(1S,2S,3S,5R)-2,6,6- | |
| | trimethylbicyclo[3.1.1]hept-3- | |
| | yl]imino}methyl)amino]phenyl}propanamide | |
| 95 | N-[4-({(Z)-(cyclohexylimino)[(3S)-3-methylpiperazin- | 517.5 |
| | 1-yl]methyl}amino)phenyl]-3-(2,4- | |
| | dichlorophenyl)propanamide | |
| 96 | (3R)-3-amino-N-{4-[((Z)-[(3S)-3-methylpiperazin-1- | 517.7 |
| | yl]{[(1S,2S,3S,5R)-2,6,6-trimethylbicyclo[3.1.1]hept- | |
| | 3-yl]imino}methyl)amino]phenyl}-3- | |
| | phenylpropanamide | |
| 97 | (2R)-2-amino-3-(2,4-dichlorophenyl)-N-{4-[((Z)- | 600.6 |
| | [(3R,5S)-3,5-dimethylpiperazin-1- | |
| | yl]{[(1S,2S,3S,5R)-2,6,6-trimethylbicyclo[3.1.1]hept- | |
| | 3-yl]imino}methyl)amino]phenyl}propanamide | |
| 98 | (3R)-N-{4-[((Z)-[(3R,5S)-3,5-dimethylpiperazin-1- | 543.8 |
| | yl]{[(1S,2S,3S,5R)-2,6,6-trimethylbicyclo[3.1.1]hept- | |
| | 3-yl]imino}methyl)amino]phenyl}-1,2,3,4- | |
| | tetrahydroisoquinoline-3-carboxamide | |
| 99 | (2R)-2-(acetylamino)-N-{4-[((Z)-[(3R,5S)-3,5- | 605.8 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}-3-(2-fluoro-4- | |
| | methylphenyl)propanamide | |
| 100 | (2S)-2-aqmino-3-(21,4-dichlorophenyl)-N-{4-[((Z)- | 600.6 |
| | [(3R,5S)-3,5-dimethyl]piperazin-1- | |
| | yl]{[(1S,2S,3S,5R)-2,6,6-trimethylbicyclo[3.1.1]hept- | |
| | 3-yl]imino}methyl)amino]phenyl}propanamide | |
| 101 | (2R)-2-(acetylamino)-N-{4-[((Z)-[(3R,5S)-3,5- | 621.8 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}-3-[2-fluoro-4- | |
| | (methyloxy)phenyl]propanamide | |
| 102 | (2S)-2-(acetylamino)-N-{4-[((Z)-[(3R,5S)-3,5- | 621.8 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}-3-[2-fluoro-4- | |
| | (methyloxy)phenyl]propanamide | |
| 103 | (2R)-2-(acetylamino)-3-(1,3-benzodioxol-4-yl)-N-{4- | 617.8 |
| | [((Z)-[(3R,5S)-3,5-dimethylpiperazin-1- | |
| | yl]{[(1S,2S,3S,5R)-2,6,6-trimethylbicyclo[3.1.1]hept- | |
| | 3-yl]imino}methyl)amino]phenyl}propanamide | |
| 104 | (2R)-2-(acetylamino)-3-(2,4-difluorpphenyl)-N-(4- | 609.8 |
| | [((Z)-[(3R)5S)-3,5-dimethylpiperazin-1- | |
| | yl]{[(1S,2S,3S,5R)-2,6,6-trimethylbicyclo[3.1.1]hept- | |
| | 3-yl]imino}methyl)amino]phenyl}propanamide | |
| 105 | (2R)-2-(acetylamino)-N-{4-[((Z)-[(3R,5S)-3,5- | 603.8 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}-3-[3- | |
| | (methyloxy)phenyl]propanamide | |
| 106 | (2R)-2-amino-N-{4-[((Z)-[(3R,5S)-3,5- | 563.8 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}-3-(2-fluoro-4- | |
| | methylphenyl)propanamide | |
| 107 | (2R)-2-amino-N-{4-[((Z)-[(3R,5S)-3,5- | 579.8 |
| | dimethylpiperazine-1-yl]{[(1S,2S,3S,5R)-2,6,6- | |
| | trimethylbicyclo[3.1.1]hept-3- | |
| | yl]imino}methyl)amino]phenyl}-3-[2-fluoro-4- | |
| | (methyloxy)phenyl]propanamide | |
| 108 | (2R)-2-(acetylamino)-3-(1,3-benzodixol-5-yl)-N-{4- | 617.8 |
| | [((Z)-[(3R,5S)-3,5-dimethylpiperazin-1- | |
| | yl]{[(1S,2S,3S,5R)-2,6,6-trimethylbicyclo[3.1.1]hept- | |
| | 3-yl]imino}methyl)amino]phenyl}propanamide | |
| 109 | (2S)-2-(acetylamino)-3-(1,3-benzodioxol-4-yl)-N-{4- | 603.8 |
| | [((Z)-[(3S)-3-methylpiperazin-1-yl]{[(1S,2S,3S,5R)- | |
| | 2,6,6-trimethylbicyclo[3.1.1]hept-3- | |
| | yl]imino}methyl)amino]phenyl}propanamide | |
| 110 | (2S)-2-(acetylamino)-N-{4-[((Z)-[(3R,5S)-3,5- | 603.8 |
| | dimethylpiperazine-1-yl]{[(1S,2S,3S,5R)-2,6,6- | |
| | trimethylbicyclo[3.1.1]hept-3- | |
| | yl]imino}methyl)amino]phenyl}-3-[3- | |
| | (methyloxy)phenyl]propanamide | |
| 111 | (2S)-2-(acetylamino)-3-(2,4-difluorophenyl)-N-{4- | 609.8 |
| | [((Z)-[(3R,5S)-3,5-dimethylpiperazin-1- | |
| | yl]{[(1S,2S,3S,5R)-2,6,6-trimethylbicyclo[3.1.1]hept- | |
| | 3-yl]imino}methyl)amino]phenyl}propanamide | |
| 112 | 1,1-dimethylethyl (1R)-2-({4-[((Z)-[(3R,5S)-3,5- | 681.9 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}amino-1- | |
| | (naphthalen-1-ylmethyl)-2-oxoethylcarbamate | |
| 113 | 1,1-dimethylethyl (1R)-2-({4-[((Z)-[(3R,5S)-3,5- | 681.9 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}amino)-1- | |
| | (naphthalen-2-ylmethyl)-2-oxoethylcarbamate | |
| 114 | (2R)-2-amino-N-{4-[((Z)-[(3R,5S)-3,5- | 581.8 |
| | dimethylpierazin-1-yl]{[(1S,2S,3S,5R)-2,6,6- | |
| | trimethylbicyclo[3.1.1]hept-3- | |
| | yl]imino}methyl)amino]phenyl}-3-naphthalen-1- | |
| | ylpropanamide | |
| 115 | (2R)-2-amino-N-{4-[((Z)-[(3R,5S)-3,5- | 581.8 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}-3-naphthalen-2- | |
| | ylpropanamide | |
| 116 | 3-[2-fluoro-4-(methyloxy)phenyl]-N-{4-[((Z)-[(3S)-3- | 564.6 |
| | methylpiperazin-1-yl]{]4- | |
| | (trifluoromethyl)cyclohexyl]imino}methyl)amino] | |
| | phenyl}propanamide | |
| 117 | (2R)-2-amino-3-[2-fluoro-4-(methyloxy)phenyl]-N-{4- | 565.7 |
| | [((Z)-[(3S)-3-methylpiperazin-1-yl]{[(1S,2S,3S,5R)- | |
| | 2,6,6-trimethylbicyclo[3.1.1]hept-3- | |
| | yl]imino}methyl)amino]phenyl}propanamide | |
| 118 | (2R)-2-acetylamino)-3-(4-bromo-2-fluorophenyl)-N- | 670.7 |
| | {4-[((Z)-[(3R,5S)-2,6,6-trimethylbicyclo[3.1.1]hept- | |
| | 3-yl]imino}methyl)amino]phenyl}propanamide | |
| 119 | (3R)-N-[(1R)-1-[(4-chlorophenyl)methyl]-2-({4-[((Z)- | 725.4 |
| | [(3R,5S)-3,5-dimethylpiperazin-1- | |
| | yl]{[(1S,2S,3S,5R)-2,6,6-trimethylbicyclo[3.1.1]hept- | |
| | 3-yl]imino}methyl)amino]phenyl}amino)-2-oxoethyl]- | |
| | 1,2,3,4-tetrahydroisoquinoline-3-carboxamide | |
| 120 | (3R)-N-[(1R)-1-[(4-chlorophenyl)methyl]-2-({4-[((Z)- | 711.4 |
| | [(3S)-3-methylpiperazin-1-yl]{[(1S,2S,3S,5R)-2,6,6- | |
| | trimethylbicyclo[3.1.1]hept-3- | |
| | yl]imino}methyl)amino]phenyl}amino)-2-oxoethyl]- | |
| | 1,2,3,4-tetrahydroisoquinoline-3-carboxamide | |
| 121 | (2S)-N-{4-[((Z)-[(3R,5S)-3,5-dimethylpiperazin-1- | 684.9 |
| | yl]{[(1S,2S,3S,5R)-2,6,6-trimethylbicyclo[3.1.1]hept- | |
| | 3-yl]imino}methyl)amino]phenyl}-3-[2-fluoro-4- | |
| | (methyloxy)phenyl]-2- | |
| | {[(phenylmethyl)oxy]methyl}propanamide | |
| 122 | (2S)-N-{4-[((Z)-[(3R,5S)-3,5-dimethylpiperazin-1- | 594.8 |
| | yl]{[(1S,2S,3S,5R)-2,6,6-trimethylbicyclo[3.1.1]hept- | |
| | 3-yl]imino}methyl)amino]phenyl}-3-[2-fluoro-4- | |
| | (methyloxy)phenyl]-2-(hydroxymethyl)propanamide | |
| 123 | (2R)-2-(acetylamino)-3-(2,4-dimethylphenyl)-N-{4- | 601.8 |
| | [((Z)-[(3R,5S)-3,5-dimethylpiperazin-1- | |
| | yl]{[(1S,2S,3S,5R)-2,6,6-trimethylbicyclo[3.1.1]hept- | |
| | 3-yl]imino}methyl)amino]phenyl}propanamide | |
| 124 | phenylmethyl (1R)-2-({4-[((Z)-[(3S,5S)-3,5- | 727.9 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}amino)-1-{[2-fluoro-4- | |
| | (methyloxy)phenyl]methyl}-2- | |
| | oxoethyl(methyl)carbamate | |
| 125 | (2R)-2-(acetylamino)-3-(2,4-dimethylphenyl)-N-{4- | 601.8 |
| | [((Z)-[(3R,5S)-3,5-dimethylpiperazin-1- | |
| | yl]{[(1S,2S,3S,5R)-2,6,6-trimethylbicyclo[3.1.1]hept- | |
| | 3-yl]imino}methyl)amino]phenyl}propanamide | |
| 126 | (2S)-3-(2,4-dimethylphenyl)-N-{4-[((Z)-[(3R,5S)-3,5- | 574.8 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}-2- | |
| | (hydroxymethyl)propanamide | |
| 127 | (2S)-N-{4-[((Z)-[(3R,5S)-3,5-dimethylpiperazin-1- | 578.8 |
| | yl]{[(1S,2S,3S,5R)-2,6,6-trimethylbicyclo[3.1.1]hept- | |
| | 3-yl]imino}methyl)amino]phenyl}-3-(4-fluoro-2- | |
| | methylphenyl)-2-(hydroxymethyl)propanamide | |
| 128 | (2S)-N-{4-[((Z)-[(3R,5S)-3,5-dimethylpiperazin-1- | 668.9 |
| | yl]{[(1S,2S,3S,5R)-2,6,6-trimethylbicyclo[3.1.1]hept- | |
| | 3-yl]imino}methyl)amino]phenyl}-3-(2-fluoro-4- | |
| | methylphenyl)-2- | |
| | {[(phenylmethyl)oxy]methyl}propanamide | |
| 129 | (2S)-3-(2,4-dimethylphenyl)-N-{4-[((Z)-[(3R,5S)-3,5- | 664.9 |
| | dimethylpiperazin-1-yl]{[(1S,2S,3S,5R)-2,6,6- | |
| | trimethylbicyclo[3.1.1]hept-3- | |
| | yl]imino}methyl)amino]phenyl}-2- | |
| | {[(phenylmethyl)oxy]methyl}propanamide | |
| 130 | (2S)-N-{4-[((Z)-[(3R,5S)-3,5-dimethylpiperazin-1- | 578.8 |
| | yl]{[(1S,2S,3S,5R)-2,6,6-trimethylbicyclo[3.1.1]hept- | |
| | 3-yl]imino}methyl)amino]phenyl}-3-(2-fluoro-4- | |
| | methylphenyl)-2-(hydroxymethyl)propanamide | |
| 131 | (2R)-N-{4-[((Z)-[(3S,5S)-3,5-dimethylpiperazin-1- | 593.8 |
| | yl]{[(1S,2S,3S,5R)-2,6,6-trimethylbicyclo[3.1.1]hept- | |
| | 3-yl]imino}methyl)amino]phenyl}-3-[2-fluoro-4- | |
| | (methyloxy)phenyl]-2-(methylamino)propanamide | |
| 132 | (2S)-3-{[3-bromophenyl)methyl]oxy}-N-{4-[((Z)- | 763.8 |
| | [(3R,5S)-3,5-dimethylpiperazine-1- | |
| | yl]{[(1S,2S,3S,5R)-2,6,6-trimethylbicyclo[3.1.1]hept- | |
| | 3-yl]imino}methyl)amino]phenyl}-2-{[2-fluoro-4- | |
| | (methyloxy)phenyl]methyl}propanamide | |
| 133 | (2S)-N-{4-[((Z)-[(3R,5S)-3,5-dimethylpiperazin-1- | 698.9 |
| | yl]{[(1S,2S,3S,5R)-2,6,6-trimethylbicyclo[3.1.1]hept- | |
| | 3-yl]imino}methyl)amino]phenyl}-3-[2-fluoro-4- | |
| | (methyloxy)phenyl]-2-{[(4- | |
| | methylphenyl)methyl]oxy}methyl)propanamide | |
| 134 | (2S)-N-{4-[((Z)-[(3R,5S)-3,5-dimethylpiperazin-1- | 702.9 |
| | yl]{[(1S,2S,3S,5R)-2,6,6-trimethylbicyclo[3.1.1]hept- | |
| | 3-yl]imino}methyl)amino]phenyl}-4-[2-fluoro-4- | |
| | (methyloxy)phenyl]-2-({[(4- | |
| | fluorophenyl)methyl]oxy}methyl)propanamide | |
| 135 | (2S)-3-{[(4-chloro-2-fluorophenyl)methyl]oxy}-N-{4- | 737.7 |
| | [((Z)[(3R,5S)-3,5-dimethylpiperazin-1- | |
| | yl]{[(1S,2S,3S,5R)-2,6,6-trimethylbicyclo[3.1.1]hept- | |
| | 3-yl]imino}methyl)amino]phenyl}-2-{[2-fluoro-4- | |
| | (methyloxy)phenyl]methyl}propanamide | |

EC₅₀ values of test compounds were determined by treating cells expressing MC4-R with test compound and lysing the cells and measuring intercellular cAMP concentration with an Amersham-Pharmacia RPA-559 cAMP Scintillation Proximity Assay (SPA) kit. The compounds described above were synthesized and tested according to this assay. Each of the named compounds of Examples 3-135 were found or will be found to exhibit MC4-R agonist activity and thus is useful in treating MC4-R mediated conditions. Additionally, Examples 3-95, 97, 99-102, 106, 107, 121, 126, 128-130, and 132-135 exhibited −log EC₅₀ values above about 3. For these reasons, each of the exemplary compounds are individually preferred and are preferred as a group. Furthermore, the groups corresponding to R¹ through R¹⁹, R^{1,} through R^{4′}, Q, W, X, Y, and Z, and the values of m and n for each of the named compounds of Examples 3-135 are also preferred. Nomenclature for these compounds was provided using ACD/namebatch version 4.53 software available from Advanced Chemistry Development, Inc. and ACD Name version 5.07 software (Nov. 14, 2001) available from Advanced Chemistry Development, Inc. Some of the starting materials were named using standard IUPAC nomenclature and ChemDraw AutoNom version 2.1. Example compounds 3-135 are illustrative and should not be construed as limiting of the instant invention.

In Vivo Studies of MC4-R Agonists on Energy Intake, Body Weight, Hyperinsulinemia, and Glucose Levels

In vivo studies are conducted to observe the effect of MCR-4 agonists on energy intake, body weight, hyperinsulinemia, and glucose levels. All studies are conducted with male 9-10 week old ob/ob mice which display early onset of obesity, insulin resistance and diabetes due to leptin deficiency. Mice are acclimated in the facility for 1 week before studies and are caged individually. Vehicle-treated (control) and drug treated mice studies are always run in parallel. In multi-day studies, mice (8-15 per group) are monitored for baseline body weight, fasting levels of glucose, insulin, blood lipids and energy expenditure and then injected twice daily (9 a.m. and 5 p.m.) with 3 mg/kg of a MC4-R agonist of the present invention for 4 weeks. Body weight as well as food and water intake are monitored daily. Animals are fasted overnight for measurements of fasting levels of glucose, insulin, and lipids once a week until the end of the study. Energy expenditure (resting metabolic rate, i.e., O₂ consumption and CO₂ production) are monitored in air tight chambers at the end of the study on fed animals. O₂ consumption and CO₂ production are measured using Oxymax systems (Columbus Instruments). Oral glucose tolerance test (OGTT—a routine test for diabetes and glucose intolerance) is performed on overnight fasted mice at the end of the study. Blood glucose and oral glucose tolerance are measured using a glucose monitor (Onetouch sold by Lifescan). Free fatty acids are measured using an non-esterified free fatty acids enzymatic assay (Waco Chemicals). Serum Insulin levels are measured by immunoassay (Alpco).

Results

The effect of the compounds of the present invention on food intake is determined by measuring grams/mouse/day throughout a 4 week study. Food is monitored every morning. Cumulative food intake represents the total amount of grams the mice consume during the study. A significant reduction in food intake is demonstrated in those mice treated IP with the compounds of the present invention.

The effect of the compounds of the present invention on body weight is determined by measuring grams/mouse throughout a 4 week study. Mice are weighed every morning. A significant body weight reduction is demonstrated in those mice treated IP with the compounds of the present invention.

The effect of the compounds of the present invention on blood glucose levels is determined by measuring blood glucose levels as represented as mg of glucose/dL of blood. Mice are fasted overnight and glucose levels are measured the following morning. Vehicle treated mice show an increase in blood glucose consistent with the rapid progression of diabetes in this mouse strain whereas, diabetes is slowed down considerably in drug treated mice. A significant reduction in fasting glucose levels is demonstrated in those mice treated IP with the compounds of this invention.

The effect of the compounds of the present invention on glucose levels during oral glucose tolerance test (OGTT) is determined by measuring blood glucose in overnight fasted mice. Blood glucose is represented as mg of glucose/dL of blood. Glucose levels are measured the following morning. Orally administered glucose quickly elevates blood glucose, similar to a meal, and the response to this exogenous glucose gives a measure of how well the body regulated glucose horneostasis. Vehicle treated mice show an elevated response to glucose consistent with their diabetic state, whereas drug treated mice show a very much improved glucose disposal.

The effect of the compounds of the present invention on free fatty acid (FFA) levels is determined by measuring mmoles of FFA/L of serum. Mice are fasted overnight and free fatty acid levels are measured the following morning. Vehicle treated mice show elevated levels of FFA throughout the study consistent with their obese state, whereas the drug treated mice diabetes show a dramatic decrease.

The effect of the compounds of the present invention on serum insulin levels is determined by measuring serum insulin levels one hour after single IP dosing of I and 3 mg/kg in overnight fasted ob/ob mice. Serum insulin levels are represented as ng of insulin/mL of serum. Drug treated mice show a dose dependent decrease relative to vehicle.

It is understood that the invention is not limited to the embodiments specifically set forth herein for illustration, but embraces all such forms thereof as would be understood by one of skill in the art and come within the scope of the following claims.

What is claimed is:

1. A compound of formula A¹-A²-A³-A⁴
wherein
A¹ is a group of formula IIA or IIB; 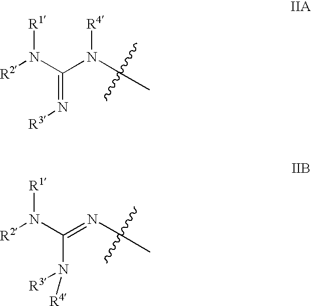
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;
or R^{1′} and R^{2′}, together with the nitrogen to which they are bound, form a substituted or unsubstituted heterocyclyl or heteroaryl group;
R^{3′} is selected from the group consisting of substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl, alkynyl, cycloalkyl, heterocyclylalkyl, cycloalkylalkyl, aryl, heteroaryl, heterocyclyl, arylalkyl, and heteroarylalkyl groups;
A² is selected from the group consisting of substituted and unsubstituted aryl groups and substituted and unsubstituted heteroaryl groups;
A³ is a covalent bond such that A² is directly bonded to A⁴, or A³ is a linking group selected from the group consisting of O, S, —NR^{a}—, —C(═O)—, —C(═O)O—, —NR^{a}C(═O)—, —SO₂NR^{a}—, —C(═S)—, —C(═O)S—, —P(═O)R^{b}—, —SO₂—, and —S(═O)—, wherein if A³ is a linking group, then it is bonded to A² and A⁴ in a configuration selected from the group consisting of A²-O-A⁴, A²-S-A⁴, A²-NR^{a}-A⁴, A²-C(═O)-A⁴, A²-C(═O)O-A⁴, A⁴-C(═O)O-A², A²-NR^{a}C(═O)-A⁴, A⁴-NR^{a}C(═O)-A², A²-SO₂NR^{a}-A⁴, A⁴-SO₂NR^{a}-A², A²-C(═S)-A⁴, A²-(C═O)S-A⁴, A⁴-(C═O)S-A², A²-(P═O)R^{b}-A⁴, A²-SO₂-A⁴, and A²-S(═O)-A⁴ provided that if A³ is a linking group with the configuration A⁴-NR^{a}C(═O)-A², then A² is not a substituted or unsubstituted phenyl group and is not a substituted or unsubstituted 6-membered N-containing heteroaryl group;
A⁴ is selected from the group consisting of substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups;
R^{a} is selected from the group consisting of H, and substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups;
R^{b} is selected from the group consisting of substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups; and
prodrugs thereof, pharmaceutically acceptable salts thereof, stereoisomers thereof, tautomers thereof, hydrates thereof, hydrides thereof, or solvates thereof.

2. The compound of claim 1, wherein A² is selected from the group consisting of substituted and unsubstituted phenyl groups and substituted and unsubstituted pyridyl groups.

3. The compound of claim 1, wherein A³ is a linking group bonded to A² and A⁴ in a configuration selected from the group consisting of A²-NR^{a}-A⁴, A²-C(═O)-A⁴, A²-C(═O)O-A⁴, A⁴-C(═O)O-A², A²-NHC(═O)-A⁴, A²-SO₂NH-A⁴, and A²-SO₂-A⁴.

4. The compound of claim 1, wherein R^{3′} is selected from the group consisting of substituted and unsubstituted cycloalkyl, polycyclic cycloalkyl, alkenyl, alkyl, and aryl groups.

5. The compound of claim 1, wherein R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups.

6. The compound of claim 1, wherein R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, cyclohexylmethyl, isopinocampheyl, 7,7-dimethylnorbornyl, 4-isopropylcyclohexyl, and 3-methylcycloheptyl groups.

7. The compound of claim 1, wherein R^{1′} is H and R^{2′} is selected from the group consisting of substituted and unsubstituted alkyl, arylalkyl, and heteroarylalkyl groups.

8. The compound of claim 1, wherein R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups.

9. The compound of claim 1, wherein R^{1′} and R^{2′} may be the same or different and are each independently selected from the group consisting of substituted and unsubstituted alkyl, arylalkyl, and heteroarylalkyl groups.

10. The compound of claim 1, wherein R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups.

11. The compound of claim 1, wherein R^{1′} and R^{2′}, together with the nitrogen to which they are bound, form a substituted or unsubstituted heterocyclyl group.

12. The compound of claim 1, wherein R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound.

13. The compound of claim 1, wherein R^{1′} and R^{2′}, together with the nitrogen to which they are bound, form a substituted or unsubstituted piperazino, morpholino, pyrrolidino, piperidino, homopiperazino, or azepino group.

14. The compound of claim 1, wherein R^{1′} and R^{2′}, together with the nitrogen to which they are bound, form a piperazino group optionally substituted by one or two methyl groups.

15. The compound of claim 1, wherein R^{a} is H.

16. The compound of claim 1, wherein A³ is a covalent bond.

17. The compound of claim 1, wherein A⁴ is a 2,4-disubstituted phenylethyl group or an indolylethyl group.

18. The compound of claim 1, wherein A⁴ is selected from the group consisting of 2,4-dihalophenylethyl, and 2,4-dialkylphenylethyl groups.

19. The compound of claim 1, wherein A⁴ is selected from the group consisting of phenylethyl, 2,4-dichlorophenylethyl, 4-methoxyphenylethyl, 4-bromophenylethyl, 4-methylphenylethyl, 4-chlorophenylethyl, 4-ethylphenylethyl, cyclohexenylethyl, 2-methoxyphenylethyl, 2-chlorophenylethyl, 2-fluorophenylethyl, 3-methoxyphenylethyl, 3-fluorophenylethyl, thienylethyl, indolylethyl, 4-hydroxyphenylethyl, 3,4-dimethoxyphenylethyl, 2-chloro-4-iodophenylethyl, 2-fluoro-4-methylphenylethyl, 2-fluoro-4-bromophenylethyl, 2-fluoro-4-methoxyphenylethyl, 2-trifluoromethyl-4-fluorophenylethyl, 2,4-difluorophenylethyl, 2,4-dimethylphenylethyl, or 2,4-dimethoxyphenylethyl groups.

20. A compound of formula I 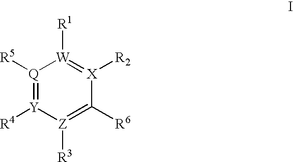
wherein
Q, W, X, Y, and Z are independently selected from the group consisting of carbon atoms and nitrogen atoms;
R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB; 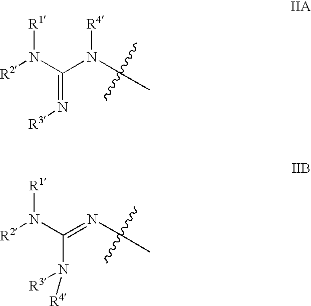
wherein R¹ may be absent if W is a nitrogen atom;
wherein R² may be absent if X is a nitrogen atom;
wherein R³ may be absent if Z is a nitrogen atom;
wherein R⁴ may be absent if Y is a nitrogen atom;
wherein R⁵ may be absent if Q is a nitrogen atom;
wherein one of R¹, R², R³, R⁴, or R⁵ is a group having the formula IIA or IIB;
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;
or R^{1′} and R^{2′}, together with the nitrogen to which they are bound, form a substituted or unsubstituted heterocyclyl or heteroaryl group;
R^{3′} is selected from the group consisting of substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl, alkynyl, cycloalkyl, heterocyclylalkyl, cycloalkylalkyl, aryl, heteroaryl, heterocyclyl, arylalkyl, and heteroarylalkyl groups;
R⁶ is a group of formula IIIA, IIIB, IIIC, IIID, or IIIE 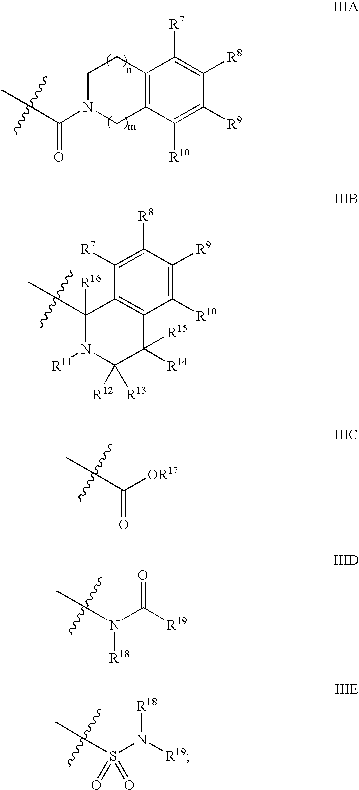
m is an integer selected from 0, 1, or 2;
n is an integer selected from 0, 1, or 2;
R⁷, R⁸, R⁹, and R¹⁰ may be the same or different and are independently selected from the group consisting of H, Cl, l, F, Br, OH, NH₂, CN, NO₂, and substituted and unsubstituted alkoxy, amino, alkyl, aryl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, and heteroarylaminocarbonyl groups;
R⁷ and R⁸ may join together with the carbon atoms to which they are attached to form a substituted or unsubstituted 5 or 6 membered ring;
R¹¹ is selected from the group consisting of H, and substituted and unsubstitued alkyl groups;
R¹², R¹³, R¹⁴, and R¹⁵ may be the same or different and are each independently selected from the group consisting of H, Cl, I, F, Br, OH, NH₂, CN, NO₂, and substituted and unsubstituted alkoxy, amino, alkyl, aryl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, and heteroarylaminocarbonyl groups;
R¹² and R¹⁴ may represent a second bond between the carbon bonded to R¹² and the carbon bonded to R¹⁴ such that the bond between the carbon bonded to R¹² and the carbon bonded to R¹⁴ is a double bond; and
R¹⁶ is selected from the group consisting of H, and substituted and unsubstituted alkyl groups;
R¹¹ and R¹⁶ may represent a second bond between the carbon bonded to R¹⁶ and the nitrogen bonded to R¹¹ such that the bond between the carbon bonded to R¹⁶ and the nitrogen bonded to R¹¹ is a double bond;
R¹⁷ is selected from the group consisting of H, and substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups;
R¹⁸ is selected from the group consisting of H, and substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups;
R¹⁹ is selected from the group consisting of substituted and unsubstituted arylalkyl, heteroarylalkyl, aryl, heteroaryl, heterocyclyl, cycloalkyl, heterocyclylalkyl, cycloalkylalkyl, alkenyl, alkynyl, and alkyl groups; and
prodrugs thereof, pharmaceutically acceptable salts thereof, stereoisomers thereof, tautomers thereof, hydrates thereof, hydrides thereof, or solvates thereof.

21. The compound of claim 20, wherein R⁶ has the formula IIIA.

22. The compound of claim 21, wherein m is 0 and n is 2.

23. The compound of claim 21, wherein m is 1 and n is 1.

24. The compound of claim 21, wherein m is 0 and n is 1.

25. The compound of claim 21, wherein m is 2 and n is 1.

26. The compound of claim 20, wherein R⁶ has the formula IIIB.

27. The compound of claim 26, wherein R¹¹ and R¹⁶ represent a second bond between the carbon bonded to R¹⁶ and the nitrogen bonded to R¹¹ such that the bond between the carbon bonded to R¹⁶ and the nitrogen bonded to R¹¹ is a double bond.

28. The compound of claim 26, wherein R¹¹ is H or a substituted or unsubstituted alkyl group and R¹⁶ is H.

29. The compound of claim 26, wherein at least one of R⁸ or R⁹ is selected from the group consisting of Br, Cl, F, I, substituted and unsubstituted alkyl groups, and substituted and unsubstituted alkoxy groups.

30. The compound of claim 20, wherein R⁶ has the formula IIIC.

31. The compound of claim 20, wherein R⁶ has the formula IIID.

32. The compound of claim 20, wherein R⁶ has the formula IIIE.

33. The compound of claim 20, wherein R⁶ has the formula IIID or IIIE and R¹⁸ is H.

34. The compound of claim 20, wherein R⁶ has the formula IIIC, IIID, or IIIE wherein R¹⁷ or R¹⁹ is selected from the group consisting of substituted and unsubstituted arylalkyl groups, and substituted and unsubstituted heteroarylalkyl groups.

35. The compound of claim 34, wherein R¹⁷ or R¹⁹ is a substituted or unsubstituted phenylalkyl group or a substituted or unsubstituted indolylalkyl group.

36. The compound of claim 34, wherein R¹⁷ or R¹⁹ is a 2,4-disubstituted phenylethyl group or an indolylethyl group.

37. The compound of claim 34, wherein R¹⁷ or R¹⁹ is selected from the group consisting of 2,4-dihalophenylethyl, and 2,4-dialkylphenylethyl groups.

38. The compound of claim 34, wherein R¹⁷ or R¹⁹ is selected from the group consisting of phenylethyl, 2,4-dichlorophenylethyl, 4-methoxyphenylethyl, 4-bromophenylethyl, 4-methylphenylethyl, 4-chlorophenylethyl, 4-ethylphenylethyl, cyclohexenylethyl, 2-methoxyphenylethyl, 2-chlorophenylethyl, 2-fluorophenylethyl, 3-methoxyphenylethyl, 3-fluorophenylethyl, thienylethyl, indolylethyl, 4-hydroxyphenylethyl, 3,4-dimethoxyphenylethyl, 2-chloro-4-iodophenylethyl, 2-fluoro-4-methylphenylethyl, 2-fluoro-4-bromophenylethyl, 2-fluoro-4-methoxyphenylethyl, 2-trifluoromethyl-4-fluorophenylethyl, 2,4-difluorophenylethyl, 2,4-dimethylphenylethyl, or 2,4-dimethoxyphenylethyl groups.

39. The compound of claim 31, wherein R¹⁹ is a substituted arylalkyl group, and the alkyl group of the R¹⁹ arylalkyl group is substituted with an amino or acetamido group.

40. The compound of claim 20, wherein Q is a carbon atom and R⁵ has the formula IIA or IIB.

41. The compound of claim 20, wherein Q, W, X, Y, and Z are all carbon atoms.

42. The compound of claim 20, wherein one of Q, W, X, Y, or Z is a nitrogen atom.

43. The compound of claim 20, wherein R^{4′} is an H.

44. The compound of claim 20, wherein R^{3′} is selected from the group consisting of substituted and unsubstituted cycloalkyl, polycyclic cycloalkyl, alkenyl, alkyl, and aryl groups.

45. The compound of claim 20, wherein R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups.

46. The compound of of claim 20, wherein R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, cyclohexylmethyl, isopinocampheyl, 7,7-dimethylnorbornyl, 4-isopropylcyclohexyl, and 3-methylcycloheptyl groups.

47. The compound of claim 20, wherein R^{1′} is H and R^{2′} is selected from the group consisting of substituted and unsubstituted alkyl, arylalkyl, and heteroarylalkyl groups.

48. The compound of claim 20, wherein R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups.

49. The compound of claim 20, wherein R^{1′} and R^{2′} may be the same or different and are each independently selected from the group consisting of substituted and unsubstituted alkyl, arylalkyl, and heteroarylalkyl groups.

50. The compound of claim 20, wherein R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups.

51. The compound of claim 20, wherein R^{1′} and R^{2′}, together with the nitrogen to which they are bound, form a substituted or unsubstituted heterocyclyl group.

52. The compound of claim 51, wherein R¹⁷ is H or an unsubstituted alkyl group.

53. The compound of claim 52, wherein R^{3′} is a substituted cycloalkyl group or a substituted polycyclic cycloalkyl group.

54. The compound of claim 20, wherein R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound.

55. The compound of claim 54, wherein R¹⁷ is H or an unsubstituted alkyl group.

56. The compound of claim 20, wherein R^{1′} and R^{2′}, together with the nitrogen to which they are bound, form a substituted or unsubstituted piperazino, morpholino, pyrrolidino, piperidino, homopiperazino, or azepino group.

57. The compound of claim 56, wherein R¹⁷ is H or an unsubstituted alkyl group.

58. The compound of claim 20, wherein R^{1′} and R^{2′}, together with the nitrogen to which they are bound, form a piperazino group optionally substituted by one or two methyl groups.

59. A composition comprising the compound according to claim 1 and a pharmaceutically acceptable carrier.

60. A composition comprising the compound according to claim 20 and a pharmaceutically acceptable carrier.

61. A method of treating an MC4-R mediated disease, comprising administering to a subject in need thereof, the compound according to claim 1.

62. The method according to claim 61, wherein the disease is obesity or type II diabetes.

63. A method of treating an MC4-R mediated disease, comprising administering to a subject in need thereof, the compound according to claim 20.

64. The method according to claim 63, wherein the disease is obesity or type II diabetes.
